(19) United States
(12) Patent Application Publication
(10) Pub. No.: US 2024/0252564 A1
(43) Pub. Date: Aug. 1, 2024

(54) MIR-375- AND MIR-1-REGULATED COXSACKIEVIRUS B3 HAS NO PANCREAS AND HEART TOXICITY BUT STRONG ANTITUMOR EFFICIENCY IN COLORECTAL CARCINOMAS

(71) Applicant: Technische Universität Berlin, Berlin (DE)

(72) Inventors: Henry FECHNER, Luckau (DE); Ahmet HAZINI, Berlin (DE)

(21) Appl. No.: 18/196,538

(22) PCT Filed: Aug. 4, 2021
(86) PCT No.: PCT/EP2021/071767

§ 371 Date: May 12, 2023

(30) Foreign Application Priority Data
Nov. 13, 2020 (DE) 10 2020 130 072.7

(51) Int. Cl. A61K 35/768; A61P 35/00; C12N 15/86

(52) U.S. Cl. CPC A61K 35/768; A61P 35/00; C12N 15/86; C12N 2770/32322; C12N 2770/32332; C12N 2770/32343; C12N 2770/32345; C12N 2770/32371

(57) ABSTRACT

The present invention related to an infectious complementary DNA (cDNA) construct characterized in that the cDNA comprises: —the cDNA of the CVB3 genomic RNA sequence of a Coxsackievirus B3 (CVB3); —at least one or more microRNA target sequences (miR-TS), which are complementary to one or more microRNAs having tissue-specific expression pattern, wherein the at least one or more miR-TS are integrated immediately adjacent of the 5′UTR and/or the 3′UTR of the CVB3 protein coding sequence.

### FIELD OF THE INVENTION

The invention refers to new miR-375- and miR-1-regulated Coxsackievirus B3 (CVB3) strains derived from the CVB3 Nancy strain for use in medicine.

### BACKGROUND OF THE INVENTION

Oncolytic viruses (OV) represent new effective therapeutics for many different types of cancer. The anti-tumor effectiveness of oncolytic viruses results from two different closely linked mechanisms, the induction of tumor cell lysis as a result of viral replication in the tumor cells and in a subsequent step the induction of a cytotoxic anti-tumoral immune response, leading to immunogenic tumor cell death. Oncolytic activity has been shown for several DNA and RNA 10 viruses and three of them, Rigvir (Donirna et al. 2015), Oncorine (Garber 2006) and T-Vec (Andtbacka et al. 2015), have been approved in different countries and are commercially available as oncolytic treatments for different kinds of cancer. Beyond these, viruses of several other families have already been investigated in clinical trials and some of them are expected to be approved in the near future for clinical use.

Picornaviruses are among the best investigated viruses due to their importance in variety of mammalian diseases. Since the recent development of oncolytic virotherapy, these viruses are being exploited as powerful tools for cancer treatment. Several members of the Picornaviridae family have been evaluated for the oncolytic activity in pre-clinical and clinical studies. Among them, Seneca Valley virus, Theiler's murine encephalomyelitis virus and mengovirus have been analyzed in pre-clinical investigations. Engineered oncolytic poliovirus PVSRIPO and Coxsackievirus (CV) A21 have been extensively tested in clinical trials. Picornaviruses have some advantages for use in cancer therapy. In particular, their small size, rapid replication (within 6 to 8 h), high number of progeny and induction of a strong immune response are all beneficial for cancer therapy. Moreover, picornaviruses have a comparatively small RNA genome of <10 kb, which can be transcribed in vitro and used for the generation of easy-to-engineer, infectious cDNA clones. Coxsackievirus group B serotype 3 (CVB3), a single-stranded RNA virus is a member of the picornavirus family and has been recently shown to harbor potent oncolytic activity of human lung, endometrial and colorectal carcinomas in vivo in several mouse tumor models (e.g. (Miyamoto et al. 2012; Hazini et al. 2018). Thus, CVB3s represent promising candidates for potential new effective therapeutics for many different types of cancer. CVB3 has a single-stranded positive-sense 7.4 kb length RNA genome, which consists of a 5′ untranslated region (UTR) followed by a region, which encodes for a monocistronic polyprotein and a 3′UTR (Fechner et al. 2011).

Oncolytic viruses (OVs) as CVB3 are classically defined to be naturally occurring or engineered viruses that selectively replicate in tumor cells without harming normal cells. The aforementioned definition of OVs is reflecting the theoretical ideal situation. However, in stark contrast to that, in reality, in several studies of the prior art it has been found that a majority of the oncolytic CVB3 strains induce side effects in animal or human subjects in vivo infected with those viruses. This seems to be mainly caused due to leaky infection of off-target sites as non-tumorous cells and native tissues by the virus.

In consequence, a number of CVB3 strains have been characterized by their tissue tropism and organ toxicity in order to better understand virus-host interaction and pathogenesis caused by viral infection. Among these CVB3 strains, there are strains which are highly cardiotropic, such as CVB3 H3, 31-1-31, M2, HA or H310A1, whereas a number of other strains have been found to be low-cardiotropic, e.g., Nancy. There are also CVB3 strains that preferentially infect the liver. Moreover, almost all known CVB3 strains are able to infect the pancreas. Promisingly, quite recently, the group of the inventors of the present invention demonstrated that the laboratory strain CVB3 variant PD, which derives from the Nancy strain, was shown to not infect the pancreas or heart in vivo in mice studies, while promisingly still showing reliable anti-tumor-activities. However, as shown by various different studies of different scientists—with the exception of CVB3 PD—all CVB3 variants caused at some level side effects at off-target sites in vivo in mice. This included in particular infection of the pancreas and the heart ranging from slight replication and tissues damage in these non-tumorous native individual organs to severe inflammation of the pancreas and the heart and fatal disease (Miyamoto et al. 2012; Hazini et al. 2018). These observations are in line with the finding that the pancreas is the most susceptible organ for CVB3 in mice and primary site of CVB3 infection from which the virus spread to other organs, e.g., the heart. Infection of both organs (pancreas and heart) is not restricted to animals, as CVB3 can also infect the human pancreas, leading to pancreatitis and diabetes-like symptoms, and the heart, leading to myocarditis and cardiomyopathies.

CVB3 has been widely used in in vitro or pre-clinical experimental set ups. In human patients infected with certain CVB3 variants (PD variant was not tested yet) it has been reported that these develop mild disease with influenza-like symptoms. However, unfortunately, under certain circumstances systemic infection can occur, leading to myocarditis, pancreatitis and aseptic meningo-encephalitis. In addition, some CVB3s are associated with the development of inflammatory and dilated cardiomyopathies in humans (Andreoletti et al. 2009) or CVB3 has been described in connection with severe infections of children. Thus, it seemed questionable to what extent CVB3 strains could actually be considered a safe OV. Commonly, in all these different in vivo studies with different CVB3 variants the pancreas and the heart represent the organs most strongly detrimentally affected by the virus as off-target sites. Thus, there is a major interest and need to improve the selectivity and thereby also the safety of such OVs for medical utilisation to treat subjects in need thereof.

For RNA viruses like CVB3, “miR-mediated virus detargeting” is a very effective technology, which is principally able to limit virus replication quite specifically to the tumors, thus increasing tumor selectivity of oncolytic viruses (Kelly et al. 2008). This method is based upon microRNA induced virus genome silencing. MiRNAs (abbreviated “miR” in the following) are non-coding, ˜22 nt double-stranded RNA molecules, which are processed from an imperfect ˜70-80 nucleotide (nt) stem-loop precursor. Many sequences of the mature miRNAs and their targets are (evolutionary) conserved among plants or animals. They show a so called “cross-species conservation”, e.g., miR-1 and miR-375. Many miRs are tissue or organ-specific expressed. One strand of these mature miRs, the so called “guide strand” binds to complementary cognate miR target sequences (miR-TS) through complementary base pairing in the backbone region of the genomic sequence of the virus genetically modified to be equipped with that miR-TS. Depending on the grade of sequence complementarity, i.e., grade of perfect base pair (bp) matching between the miR and its target miR-TS it either induces post-transcriptional repression of protein synthesis of the bound target sequence (less bp-match) or its catalytic degradation (high bp-match). This is permitted by guiding the RNA-induced silencing complex (RISC), which incorporates the guide strand of the miR to the complementary matching virus RNA target site (miR-TS) to the virus genome. In this method target sequences of a tissue-specifically expressed microRNA (miR), which does not occur in the tumor or is only poorly expressed/persisting in the tumor, are inserted into the virus genome. Many miRs are cell- and tissue-specifically expressed making them promising candidates for such genome silencing approaches. The insertion of miR-TS complementary to tissue-specific expressed miRs, the latter which are abundantly expressed in healthy tissues but absent or poorly expressed in cancer cells has been shown as a powerful approach to prevent undesired replication of OV in normal cells/tissues in vitro and in some few existing pre-clinical studies. In these studies by binding the miR to the miR-TS, the virus genome was degraded (high bp match) exclusively in the organs in which the miRs are expressed and thus virus replication is stopped. For plus sense single strand RNA-viruses like CVB3, this technology seems to work particularly well which may be due to the fact that the viral RNA genome is directly used as miR-target after a corresponding miR-TS has been inserted into it.

Even if the efficacy of such an approach has been reported by many studies for different viruses, for CVB3 there are only few reports known so far using miR-TS for increase of virus safety, e.g., in lung cancer entity treatment. These studies showed that insertion of miR-143TS and miR-145TS (Liu et al. 2020) into the 5′untranslated region (UTR) of the CVB3 Kandolf (Nancy strain clone) genome or insertion miR-34a/c TS into either the 5′ or the 3′ UTRs of the CVB3 Nancy genome (Jia et al. 2019), led to increase of viral safety while the viruses retained their oncolytic activity against lung carcinomas in vivo mice studies. Those miRs (miR-143, miR-145, miR-34a/c) tested are expressed in a non-tissue specific manner preferentially abundantly in normal cells or at least in the native lung tissue and are only poorly expressed in the targeted cells of the lung tumor cell lines used. Addressing prevention of undesirable CVB3 replication in the heart by insertion of heart-muscle specific miR-206 and miR-133 target sequences has been carried out with some success (He et al. 2015).

The stability of the miR-TS is an important factor contributing to the safety of miR-regulated oncolytic viruses (Kelly et al. 2008).

Moreover, in vivo after infection individual virus clones isolated from virus-infected tumors may acquire nucleotide substitutions during virus replication as reported before. Thus, the occurrence of mutations in the miR-TS, indeed represents a potential risk for viral safety. Moreover, additionally partial or complete deletions of the miR-TS can happen during replication, e.g., due to possible formation of loop structures of the miR-TS insertion, which therefore might be lost during virus replication by skipping out the region of miR-TS duringthe replication. When incorporating multiple identical or highly identical miR-TS copies (tandem repeats) the probability of the formation of such secondary loop structures of the miR-TS insertions will increase. In turn the probability of such skipping out effect of single up to the whole incorporated miR-TS copies of the tandem repeats will disadvantageously increase. It should also be mentioned that detrimentally mutations within a miR-TS may create binding sites for another miRs, which may cause unwanted and maybe serious side effects. Despite such an event has never been observed to date, it must be taken into account for further investigations and improvements. To reduce such risks a potential strategy is to increase the copy number of miR-TS in the virus backbone as it statistically increases the probability to have some copy numbers intactly expressed. On the other hand, additional miR-TS copies increase the genome size, which may potentially impair virus packaging and thereby viral replication, thus ultimately compromising its oncolytic activity, i.e., the anti-tumor efficacy of the virus. Thus, miR-TS copy number must be well balanced against the increase in the size of the genome.

In this regard in trying to solve the problem of the side effects of their oncolytic CVB3, (Jia et al. 2019) inserted four tandem copies of a target sequence of miR-34a (miR-34aTS) in the 5′ or the 3′UTR in the CVB3 genome. As a result of this modification, the virus exhibited reduced, but still detectable toxicity in mice, while the anti-tumor effectiveness of the virus construct was still retained. They also inserted each 4 tandem copies of the miR-34a into the 5′UTR and the 3′UTR. In this case reduced virus induced toxicity was observed. In another study by (Liu et al. 2020) copies of miR-TS of the tumor suppressor miR-145 and copies of the tumor suppressor miR-143 were inserted into the 5′UTR of another CVB3 variant. The virus was able to reduce tumor growth, while the toxicity of the virus was reduced. However, the virus was detrimentally still detectable, i.e., replicating and persisting in the pancreas and heart. The inventors of the present invention believe by best knowledge—without being bound by that theory—that the underlying reason for that outcome may be at least partially be based on the fact that all those tested microRNAs are tumor suppressor microRNAs and tumor suppressor microRNAs in more general seem to be not sufficiently highly expressed in specific-tissues as pancreas and heart to eliminate virus-constructs fused with the respective miR-TS. Therefore, a complete inhibition of the virus replication seems not to be feasible in those state of the art approaches without extending the copy number of those tumor suppressor microRNAs in the CVB3 variant construct. The increase of the copy number in turn gives rise to general replication impairment of the virus-construct and thus disadvantageously causes impairment of the anti-tumor effectiveness of those CVB3-miR-TS fused constructs

However, in these tumor studies of Liu et al., 2020 the inhibition of virus replication was found to be only incomplete in pancreas and heart. Additionally, the miR-143TS and miR-145TS were found to be nonstable, i.e., were lost during course of virus treatment due to deletions, which happened in the miR-TS in course of virus replication. Therefore, severe side effects occurred in the studies, as cardio- and pancreatotoxicity and positive VP1 staining in the heart and pancreas of mice that died between day 35 and 56 post-treatment with miR-143/miR-145TS containing CVB3. Similarly, in the study of Jia et al., 2019 a certain toxicity after insertion of the tumor suppressor miR-34a or miR-34c TS in the pancreas and the heart, respectively. When eight copies of a miR-34aTS were inserted into the 5′UTR (4 copies) and 3′ UTR (4 copies) toxicity was low. The disadvantage of increasing the miR-TS copy number in the genetic virus construct is seemingly that this gradually leads to a disturbance of the virus replication. In (Jia et al. 2019) it was, e.g. shown that when increasing the miR-TS copy number up to eight this disturbed the virus replication compared to a wildtype virus or a CVB3 with 4 miR-TS (e.g. FIG. 4C of (Jia et al. 2019)). Thus, giving rise to speculate that the gradual increase of miR-TS copy number could have a negative effect on virus replication when applied therapeutically in the tumor of a subject in need thereof and thus also will have detrimental consequences on the anti-tumor effectiveness of those CVB3-miR-TS fused constructs. Moreover, events of skipping out of the incorporated miR-TS may statistically increase due to the increased number of incorporated identical miR-TS sequences (tandem repeats). It is therefore an object of the present invention to overcome the described disadvantages of the state of the art OVs in the treatment of cancer, especially with respect to efficiency and safety of used OVs. Moreover, it is the aim of the present invention to provide a new variant of CVB3 with substantially retained or even improved oncolytic efficiency and improved safety compared to known strains such as e.g., CVB3 strain Nancy.

This object has been solved by an infectious cDNA construct according to claim 1. Further embodiments refer to variations of this newly developed infectious cDNA construct as well as to the viral vector particle comprising that infectious cDNA construct. Additionally, pharmaceutical composition comprising said infectious cDNA construct or said viral particle is provided as well as the use of said infectious cDNA construct or said viral particle or said pharmaceutical composition for use in treatment of cancer and/or metastasizing cancer in a subject in need thereof.

In particular, to achieve the stated goal, the present invention provides an infectious complementary DNA (cDNA) construct characterized in that the cDNA comprises:
the CVB3 genomic sequence of a Coxsackievirus B3 (CVB3);
at least one or more microRNA target sequences (miR-TS), which are complementary to one or more microRNAs having tissue-specific expression pattern,
wherein the at least one or more miR-TS are integrated immediately adjacent of the 5′UTR and/or the 3′UTR of the CVB3 protein coding sequence.

In the sense of the present invention a “infectious cDNA construct” may be understood as to include any reverse transcribed viral nucleic acid, e.g., the complete complementary DNA (cDNA) of the reverse transcribed viral RNA genome) or a sufficient part thereof to permit generation of a lytic response in cancer cells infected with said construct and/or generation of new viruses, which then will cause a lytic response in the cell and be release ready for the infection of further—sometimes—neighboring cells.

The genomic RNA of a CVB3 is a plus-strand RNA. In the sense of the present invention the term “plus-strand” means that the genomic sequence is oriented forward relative to the translation start of the coding sequence. Thus, for the construction of the infectious cDNA construct this sequence was reverse transcribed, amplified by PCR to generate a DNA double strand. Preferably, this double stranded cDNA was cloned into a plasmid to receive a cDNA version of the plus strand sequence in the infectious cDNA construct.

In the sense of the present invention “a at least one or more microRNA target sequences (miR-TS) complementary to miR sequences” is to be understood as a sequence complementary identical (100% complementary) to the at least one or several different miR strands, which allows—if the respective targeted tissue-specific miR is expressed in the infected off-target site host tissue cell—that the respective complementary miR guide strand of the host cell can bind to the miR-TS and RNA interference/RNA silencing machinery act, thus prohibiting virus replication.

In an alternative embodiment of the present invention the at least one or more microRNA target sequences (miR-TS) are “substantially” complementary to miR sequences. In the sense of the present invention “substantially complementary to miR sequences” is to be understand that the respective nucleotide sequence is at least 70% or 75% complementary, 80% complementary, more preferably, at least 90% complementary, and most preferably at least 95%, at least 96%, at least 97%, at least 98% or at least 99% complementary to the target miR sequence, both of which bind to one another by Watson & Crick base pairing.

In the sense of the present invention the three prime untranslated region (3′-UTR) is the section of messenger RNA (mRNA) or cDNA of the mRNA that immediately follows downstream (5′ to 3′) the translation termination codon and the five prime untranslated region (5′-UTR) is the section of messenger RNA (mRNA) or cDNA of the mRNA that immediately follows upstream (5′ to 3′) the start codon of a messenger RNA (mRNA) or cDNA sequence.

According to an embodiment of the infectious cDNA construct of the present invention the infectious cDNA construct is in the form of a double stranded circular molecule, preferably in the form of a plasmid. A plasmid advantageously enables a stable replicable vehicle for the infectious cDNA construct.

For the intended purpose, e.g., for oncolytic cancer treatment of subjects in need thereof there is typically a certain amount of oncolytic virus necessary. In contrast to the present invention the state of the art uses viral particles equipped with viral RNA/DNA for therapeutic purposes. According to the prior art procedure for producing OVs such CVB3s are carrying a certain modified or naturally occurring nucleic acid construct, which originates from one virus clone and which is multiplied by standard procedures to receive enough CVB3 particles in vitro, e.g., for medical treatment.

In general, CVB3s replicate with high mutation rate and there are multiple cellular infection events and thus rounds of replication necessary to end up with enough virus particles. In consequence, with a high statistical probability, there are always individual virus mutants that exist and multiply in parallel—next to the main virus clone variant carrying copies of the original starting viral cDNA. This results in the detrimental situation that the harvested CVB3 virus solution intended for therapeutic treatment is not a homogeneous solution containing only copies of the intended multiplied original virus clone but is rather a heterogenous mixture containing next to the intended clone also a subset of multiplied genetic virus clone mutants, probably with a very different genetic subset. Injecting these virus mutants may cause depending on the mutation(s) mild to severe detrimental effects to a subject, which is treated with such a heterogenous virus solution. Additionally, since the whole replication runs at random, it could be that different virus solution batches contain very different mixtures of virus particle mutants in addition to the main virus variant. This may further disadvantageously reduce the significance of such therapeutic studies using such batches as some of the subjects will most likely be treated with a different batch of the virus solution than other subjects, i.e., may receive not the same infectious starting material and the amount of copies of the intended main virus may also most likely be relevant different between different virus solution batches. Additionally, depending on the treatment regime applied it may be necessary that one and the same subject in need thereof has to be treated more than one time or has to receive multiple doses of the CVB3 virus solution, which eventually would mean that it may not receive virus solutions from one and the same batch. Thus, treated subjects would receive solutions with a mixture of quite different viruses populations perse. This also increases the probability to receive virus subsets that may cause mild to severe detrimental side effects in subjects injected with those heterogeneous virus batch solutions and reduce the significance of such clinical treatment data.

In contrast to the utilization and error prone multiplication of a virus particle carrying an infectious cDNA construct the inventors of the present invention provide a different approach and utilize an infectious cDNA construct perse.

This brings multiple advantages: Classical CVB3 viruses normally carry a single plus sense RNA strand with a positive polarity, i.e., the reading direction corresponds to that of a cellular mRNA. The nucleic acid can be used both as a genetic memory and as a template for translation. After infection, CVB3 generates a minus-strand RNA intermediate, from which multiple copies of viral plus-strand RNA copies are transcribed catalyzed by an RNA-dependent RNA polymerase, which is needed for classical virus replication in host cells. The RNA-dependent RNA polymerase lacks RNA proofreading activity and therefore mistakes introduced into the copied polynucleotide are not corrected. The frequency of errors is roughly a single nucleotide insertion error for every 10 thousand nucleotides (1×10⁻⁴), which is a bit less than the approximate length of the CVB3 vRNA. Hence, nearly every newly produced CVB3 particle has a vRNA sequence that is different from other CVB3 virus particles. In the classical state of the art approaches to multiply OVs utilizing virus particles these bearing the viral minus-strand anti-genome cDNA. After infection of the CVB3 into host cells the cDNAs are transcribed into multiple copies of viral plus-strand RNA catalyzed by a host cell DNA-dependent RNA polymerase (RNA polymerase), which has a transcription error rate of ca. 1×10⁻⁸ to 10⁻⁹ up to ca. 1×10⁻¹⁰ to 10⁻¹² depending on the respective data source.

In contrast when utilizing a cDNA clone according to the present invention to be injected: Here, the necessary multiplication of the cDNA clone of interest can easily be performed under high quality standards using well established state of the art methods well known to the artisan. E.g., the cDNA transcript can be amplified by classical PCR using, e.g., a high-fidelity DNA polymerase, which contains highly accurate proof reading function and thus produces pronounced less replication errors compared with a classical replication enzymes needed for virus replication in host cells, thus guaranteeing an accurately multiplied homogeneous infectious viral cDNA pool.

Moreover, this practice also simplifies the method of generating virus particle based OVs, which is quite complicated, time and reagent consuming in vitro. It also simplifies to comply with the Good Manufacturing Practice (GMP) standards. If the infectious cDNA clone of the present invention may be intratumorally injected into the tumor cells or transported to the tumor cells by known vector viruses. Additionally, by generating virus particles comprising the infectious cDNA construct in the infected host tumor cells, the latter which when are released from the host tumor cells can in turn infect other tumor cells, e.g., those directly adjacent. Another benefit is that the production and use of infectious cDNA clones will also be considerably cheaper and more ecological as less expensive laboratory reagents and materials will be needed when compared to said classical prior art method.

(He et al. 2015) identified several important limitations related to the insertion sites of miR-TS into the CVB3 genome. In fact, virus could only be propagated from an infectious cDNA clone if miR-TS were inserted into the 5′-UTR close to the codon for translation initiation. Insertion of miR-TS into the 5′-UTR upstream (nucleotide position 249) of the core sequence of the IRES as well as insertion of miR-TS in the 3′-UTR at nucleotide positions 7387 or 7359 to 7360 were not tolerated by the virus. As a possible reason for these observations, (He et al. 2015) suggested that the miR-TS probably disturbed the higher-order (secondary) RNA structure of the viral genome (He et al. 2015). The at least one or more miR-TS are inserted either immediately downstream of the CVB3 polyprotein initiation codon [(5+)] or immediately downstream of the stop codon of the CVB3 polyprotein into the 3′UTR. Alternatively, they are inserted before the initiation codon of the polyprotein coding sequence, i.e., in the 5′UTR.

According to an embodiment of the infectious cDNA construct of the present invention the at least one or more miR-TS are incorporated between the stop codon of the coding sequence of the 3D polymerase and the 3′UTR of the CVB3 protein encoding sequence. The at least one or more miR-TS may be integrated immediately downstream the stop codon of the coding sequence of the 3D polymerase of the CVB3 protein encoding sequence and before the 3′UTR of the CVB3 protein encoding sequence. Additionally, or alternatively, the at least one or more miR-TS are inserted immediately before (upstream 5′ to 3′) the CVB3 polyprotein initiation codon into the 5′UTR. Preferably the at least one or more miR-TS are integrated at the 3′UTR of the viral coding sequence, preferably immediately downstream (5′ to 3′) of the stop codon of the CVB3 coding sequence and immediately before the 3′UTR of the CVB3 coding sequence. Optionally, the at least one or more miR-TS are flanked by at least one stuffer sequence, each consisting of at 15 least 5 to 30 bp, at least 8 to 30 bp or at least 10 to 30 bp. The at least one or more miR-TS may be flanked monodirectional or bidirectional by a stuffer sequence. The inventors of the present invention have positively found when the at least one or more miR-TS were flanked by at least one stuffer sequence and this fusion sequence of miR-TS+stuffer sequence(s) was then integrated before the 3′UTR of the viral coding sequence, preferably immediately downstream (5′ to 3′) of the stop codon of the CVB3 coding sequence and immediately before the 3′UTR of the CVB3 coding sequence that this seems to have a positive effect on virus replication and reproduction. This seems to hold true especially for CVB3 virus strains as e.g., PD-0 and rPD, which show lower basal replication efficiency compared to the wildtype. Thus, in turn one can speculate that the lack of stuffer sequences may negatively affect virus replication and reproduction.

According to an embodiment of the infectious cDNA construct of the present invention the at least one or more miR-TS are complementary to miR sequences, which are specifically expressed in the human pancreas tissue and/or are complementary to miR sequences, which are specifically expressed in the human heart tissue.

According to an embodiment of the infectious cDNA construct of the present invention the at least one or more miR-TS are complementary to a miR sequence selected from the group consisting of human pancreas tissue specific expressed miRs: miR-375, miR-690, miR-375, miR-217, miR-216a, miR-216b, miR-200a, miR-200b, miR-200c, miR-429, miR-141 and/or human heart tissue specific expressed miRs: miR-1, mriR-133, miR-206.

According to an embodiment of the infectious cDNA construct of the present invention the at least one or more miR-TS are complementary to a miR sequence selected from the group consisting of human pancreas tissue specific expressed miR-375 and human heart tissue specific expressed miR-1. The nucleic acid sequence of the miR-375TS comprises or consists of 5′-TCACGCGAGCCGAACGAACAAA-3′ (SEQ ID No: 1). The nucleic acid sequence of the miR-1TS comprises or consists of 5′-ATACATACTTCTTTACATTCCA-3′ (SEQ ID No: 2).

Preferably the at least one or more miR-TS are integrated at the 3′UTR of the viral coding sequence, preferably immediately downstream of the stop codon (5′ to 3′) in the 3′UTR. The inventors of the present invention have found that when intratumorally injecting a virus carrying an infectious cDNA construct which carries an insertion of the miR-TS at the 3′UTR of the cDNA region of the construct, which encodes for the viral polyprotein does not markedly impair general CVB replication or CVB3 viral growth, however enables suppression of tumor cell growth infected with the cDNA construct while not effecting the pancreas or heart tissue cells (only slightly or no infectious cDNA construct could be measured) (see FIG. 4).

When using packed in virus particles the infectious cDNA constructs comprising miR-TS complementary to a miR specifically expressed in the human pancreas tissue (miR-375TS) the inventors of the present invention could clearly indicate that those infectious cDNA constructs advantageously ensure that the virus cannot efficiently replicate in native non-tumorous pancreas tissue and heart tissue in vivo in subcutaneously colorectal tumor implanted mice (see e.g., FIG. 5 C, E, K). These observations are in line with the finding that the pancreas is the most susceptible organ for CVB3 in mice and primary site of CVB3 infection from which the virus spread to other organs, e.g., the heart. Thus, by utilizing the cDNA constructs of the present invention which comprise a miR-TS complementary to miR specifically expressed in the human pancreas tissue also the heart tissue could be beneficially protected from CVB3 virus replication.

Of note this effect to protect the heart tissue from virus replication was advantageously found to be even more pronounced when using in parallel to the at least one or more miR-TS which are complementary to miR sequences, which are specifically expressed in the human pancreas tissue additionally ones which are complementary to miR sequences, which are specifically expressed in the human heart tissue (see e.g., FIG. 5 E, H).

Moreover, the inventors could show that when injecting said described naked infectious cDNA constructs (without virus capsid) comprising miR-TS complementary to miR specifically expressed in the human pancreas tissue and/or which are complementary to miR sequences, which are specifically expressed in the human heart tissue in host cells which show a low expression status of theses miRs or where these miRs were absent that these were able to replicate in those host cells and generate infectious virus particles comprising said infectious cDNA construct (cf. FIG. 2, and data not shown).

According to an embodiment of the infectious cDNA construct of the present invention the at least one or more miR-TS are present as twofold, threefold, fourfold, fivefold or more multi-fold repetitions or repetition cassettes. The repetitions can be in the form of “tandem repeats”, which is to be understand as relative short nucleotide sequences which represents a single repeat unit, the latter which is repeated and the repetition units are directly adjacent to each other or connected only with a short nucleotide spacer sequence, which separates them.

A “repetition cassette of miR-TS” according to the present invention means one miR-TS is followed by another different miR-TS, which is directed to another miR. “Followed” in the sense of the present invention means here that both miR-TS are either connected directly adjacent to each other or connected only with a short nucleotide spacer sequence, which separates them.

Both miR-TS and the eventually present spacer sequence together build up a unit of a repetition cassette. According to a preferred embodiment of the infectious cDNA construct of the present invention a repetition cassette is built up by a miR-TS complementary to a miR sequence selected from the group consisting of human pancreas tissue specific expressed miR-TS (e.g., miR-375) followed by a miR sequence selected from the group consisting of a human heart tissue specific expressed miR-TS (e.g., miR-1). According to alternative preferred embodiments of the infectious cDNA construct of the present invention a repetition cassette is built up by a twofold repetions of a miR-TS complementary to a miR sequence selected from the group consisting of human pancreas tissue specific expressed miR-TS (e.g., miR-375) followed by a single or twofold repetions of a miR sequence selected from the group consisting of a human heart tissue specific expressed miR-TS (e.g., miR-1).

A spacer sequence according to the present invention may consists of a short nucleotide (nt) sequence of, e.g., 1 to 16 nt, 3 to 10 nt, 3 to 16 nt, 3 to 15 nt, 4 to 8 nt, 4 to 6 nt or 4 to 5 nt (cf., e.g., FIG. 1 B or FIG. 6 B together with the respective Figure legend). Spacer sequences inserted between each miR-TS improve cognate mature miR-binding und thus potentially improve RNA silencing efficiency.

According to an embodiment of the infectious cDNA construct of the present invention the at least one or more miR-TS are present as at least onefold up to fourfold, at least twofold up to fourfold or preferably at least onefold up to threefold. According to a preferred embodiment of the infectious cDNA construct of the present invention the at least one or more miR-TS are present as at least twofold up to threefold repetitions or repetition cassettes.

As stated above the stability of the miR-TS is an important factor contributing to the safety of miR-regulated oncolytic viruses. However, occurrence of mutations in the miR-TS, indeed represents a potential risk for viral safety. The inventors of the present invention have found individual virus clones isolated after long term infection (32 days post initial infection) from H3N-375-infected tumors with three copies of miR-375TS and H3N-375/1-infected tumors with two copies of miR-375TS and two copies of miR-1TS may acquire nucleotide substitutions during OV treatment of mice in vivo. However, in each of such virus with miR-TS mutations only one miR-TS and only one copy of that was found to be affected by this kind of mutations, so that in H3N-375TS at least two miR-375TS and in H3N-375/1TS at least one miR-375TS and at least one miR-1TS remained intact. Thus, interestingly, indicating that a at least twofold repetition of the miR-TS might obviously sufficient to maintain the prevention of viral replication in the pancreas and 25 the heart, respectively and this even under long term OV treatment. I.e., guaranteeing genetic stability of the miR-TS of the infectious cDNA construct and thereby increasing the safety of the virus, while at the same time not increasing the genetic sequence length of the virus particle, thus not markedly effecting the virus particle replication.

According to an embodiment of the infectious cDNA construct of the present invention the expression of the backbone of the cDNA encoding the viral coding sequence is under control of a tumor specific promoter selected from the group consisting of: human telomerase reverse transcriptase promoter (hTERTp); carcinoembryonic antigen (CEA) and Tyrosinase, alpha fetoprotein (AFP) promoter, Prostata specific antigen promoter (PSA), DF3/MUC1 promoter, Tcf-responsive promoter, tyrosinase promoter, rat probasin promoter, IAI.3B promoter, osteocalcin promoter, L-plastin promoter, carcinoembryonic antigen (CEA) promoter, midkine promoter, E2F-1 promoter, HIF-1-responsive promoter, estrogen-hypoxia dual promoter and the like. By this, the replication of the infectious cDNA construct will be even more selectively restricted to tumor cells, expressing transcription factors for that promoter utilization, thus further increasing therapeutic safety of the OV.

According to an embodiment of the present invention the cDNA construct further comprises at least one or more sequence elements selected from the group consisting of: Multiple cloning site, origin of replication, and transgene, wherein these further sequences are integrated into the backbone of the cDNA construct. Transgenes may comprise a selection gene or an RNA transcript of therapeutically interesting cDNAs or other small RNAs, e.g., microRNA, shRNA, siRNAs, all which are well known by an artisan. Examples of such a transgene are immune system stimulating transgenes as interleukine 2 (IL-2), IL-4, IL-6, IL-12, IL-18, IL-24, IFN-α, IFN-β, IFN-γr granulocyte colony-stimulating factor (G-CSF) or tumor toxic genes.

Tumor toxic genes are to be understood to reveal toxicity one's expressed in tumor cells and are selected from the group consisting of tumorsuppressor genes as p53, adenomatous polyposis coli tumour suppressor gene and BRCA1, suicide genes as cytosine deaminase (CD) and herpes simplex virus 1 thymidine kinase (HSV1-TK), apoptose inducing genes as Tumor Necrosis Factor Related Apoptosis Inducing Ligand (TRAIL) and Fas Ligand (FasL), tumor-associated antigens (TAAs) and neo antigens and angiogenesis inhibitors such as angiostatin, thrombospondin, platelet factor 4, and hepatocyte growth factor antagonist NK4. According to another embodiment the infectious cDNA construct of the present invention and/or the genomic sequence of the CVB3 group virus encodes a replication competent CVB3 virus and/or a vector derived viral particle.

Principally the “Coxsackievirus B3 group (CVB3)” according to the present invention may be any Coxsackievirus B3 group virus including known and classified CVB3 viruses and yet to be classified CVB3 viruses. The CVB3 may be selected from the group consisting of both prototype and clinically isolated viruses. It may be naturally occurring or a “modified form thereof”. The Coxsackie B3 group virus is “naturally-occurring” when it can be isolated from a source in nature and has not been intentionally modified in the laboratory (“modified form”)—for instance the CVB3 may be obtained from a human patient.

In WO2019/063838A1 the inventors of the present invention identified CVB3 strain PD-0 to be a promising OV, as it does not replicate in any off-target site including heart and pancreas when administered in vivo to mice, while pertaining its anti-tumor efficiency. Thus, according to a preferred embodiment of the infectious cDNA construct of the present invention the genomic sequence of CVB3 is selected from attenuated CVB3 group virus strains derived from the Nancy strain, PD, e.g., rPD (recombinant PD-0 cDNA clone) or a modified form thereof, as described in detail—e.g., the genomic sequence—in WO2019/063838A1, which is published and incorporated herewith by reference. CVB PD-0 comprises an exchange of the amino acid residues consisting of amino acid residue K78, A80, A91, and 192 in the viral capsid protein 1 (VP1) and both of the amino acid residue M34 and Y237 in the viral capsid protein 3 (VP3).

According to a preferred embodiment of the infectious cDNA construct of the present invention the genomic sequence of the modified CVB3 PD-0 form is a recombinant cDNA clone selected from rPD_{HiFi} and rPD-eGFP as described in detail in WO2019/063838A1.

Given heterogeneity of different CVB3 virus strains with respect to anti-tumor-activity, e.g., rPD equipped with miR-375 or miR-375/miR-1 was surprisingly found to be equally or even more effective with respect to permitting tumor cytotoxicity as compared to respective miR-TS equipped approaches using H3 virus. Also, CVB3 variants with other receptor tropism and more toxic in normal tissues than PD may be potential candidates for OV and an interesting focus. However, due to the risk of pancreatitis and myocarditis and general off-target site cell infection and lytic events their replication potential has to be massively reduced, e.g., by miR detargeting utilizing the miR-TS strategy of the present invention, e.g., miR-375TS and miR-1TS detargeting. Recently, the research group of the Applicant could prove that concept. Moreover, they found out that PD-0 (e.g. rPD) has comparable anti-tumor efficacy as the aggressive H3 CVB3 variant, when the coding sequence of the latter was fused with miR-375TS, which stopped replication of H3 virus in pancreas and thus further extra distribution of the virus to the tumor tissue (cf. (Hazini et al. 2021)). Thus, according to an embodiment of the infectious cDNA construct of the present invention the genomic sequence of CVB3 is selected from aggressive CVB3 group virus strains PD, rPD, Nancy, H3, 31-1-93, RD, P2035A, 28, HA and GA and wherein the genomic sequence of the CVB3 is defined by a nucleotide sequence of one of those strains or comprising the genomic sequence of one of those CVB3 strains.

Very promising the inventors of the present invention could show that the pathogenicity of a highly virulent CVB3 strain can be prevented by equipping the virus with miR-TS copies of the pancreas-specifically expressed miR-375 (three copies—H3N-375TS) or in another approach pancreas-specifically expressed miR-375 (two copies) and the cardiac-specifically expressed miR-1 (two copies) (H3N-375TS/1TS) in a nude mice model of human colorectal carcinoma. Moreover, three of four tested tumor bearing animals of the H3N-375TS/1TS approach promising showed viremia and one did not show any viral titer in blood (cf. FIG. 5 I). Moreover, notably, because of complete remission of three virus-infected tumors, the H3N-375TS titer of the totally four study animals treated with H3N-375TS bearing viral particles (carrying three copies of miR-375TS) was only determined in one tumor. This clearly indicates the promising strong and selective anti-tumor-activity of these miR-TS bearing recombinant H3 variants in vivo at their intended target sites (tumor), while at the same time they do not or not markedly effect off-target sites as all tested non-tumor tissues stayed markedly virus-unaffected (no virus replication detected, no virus mediated cytotoxicity detected). In sharp contrast to these findings a single dose of parental CVB3 strain H3 caused mice to become moribund four days after intratumoral virus administration, three injections of miR-TS equipped viruses H3N-375TS and H3N-375/1TS did not cause virally induced sickness. Importantly, the viruses retained their oncolytic activities in vivo after long term treatment and efficiently reduced growth of the pre-implanted colorectal carcinomas.

Further, another object of the present invention is the provision of an infectious viral particle comprising the cDNA construct according to the present invention. This viral particle can derive from a Coxackie virus, particularly a CVB3 virus, but also any other suitable vector or carrier virus.

In the sense of the present invention a “infectious viral particle” or virus is to be understood as to include the infectious cDNA construct according to the present invention to permit generation of a lytic response in virus-infected cancer cells and/or generation of new infectious viruses or viral particles, which then will cause a lytic response in the cell and upon release infect further—sometimes—neighboring cells.

According to still another aspect the present invention relates to a pharmaceutical composition comprising the infectious cDNA according to the present invention and/or the viral particle according to the present invention and a pharmaceutical acceptable carrier or diluent.

Suitable pharmaceutically acceptable excipient, diluent and carriers according to the inventive pharmacological composition are well known by an artisan. In brief, examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin orsqualane; cellulose derivatives such as methyl cellulose, ethyl cellulose, carboxymethylcellulose, sodium carboxymethylcellulose hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3butylene glycol or glycerin; fatty acid esters such as isopropyl palmitate, isopropyl myristate or ethyl oleate; polyvinylpyrolidone; agar; gum tragacanth or gum acacia, and petroleum jelly. Typically, the carrier or carriers will form from 10% to 99.9% by weight of the pharmacological compositions.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition, these oral formulations may contain suitable flavouring and colouring agents. When used in capsule form the capsules may be coated with compounds such as glyceryl monostearate or glyceryl distearate, which delay disintegration.

The pharmacological composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The present invention further incorporates the inventive Infectious cDNA construct, the inventive infectious viral particle or the inventive pharmaceutical composition for use in the treatment of cancer and/or metastasizing cancer, wherein the miR sequence with tissue-specific expression complementary to the at least one or more miR-TS is each highly expressed in said tissue or tissues as compared to the respective expression status in the cancer and/or metastasizing cancer, where the expression status is low or absent.

According to an embodiment of said infectious cDNA construct, infectious viral particle or pharmaceutical composition for use in the treatment of cancer and/or metastasizing cancer, the cancer is selected from the group consisting of colorectal cancer (colon cancer), breast cancer, lung cancer, liver cancer and/or the corresponding metastases of the aforementioned cancers.

According to a preferred embodiment of said infectious cDNA construct, infectious viral particle or pharmaceutical composition for use in the treatment of cancer and/or metastasizing cancer, the cancer is selected from the group consisting of colorectal cancer and corresponding metastases or lung cancer and corresponding metastases.

According to another embodiment of the invention the range of viral dose is between about 5×10⁵ to about 1×10⁸ plaque forming units (PFU), preferably between about 1×10⁶ to about 1×10⁷ PFU and most preferred between about 3×10⁶ to about 1×10⁷ PFU. It is also provided that the application of the viral dose is administered 2-, 3-, or 4-times depending of the size of the tumor, the patients response, the tumor regression rate and also the body weight of the patient. Typically, a dose of about 4.5×10⁶ PFU/kg for a 70-kg patient is to be considered.

For treatment purposes according to the present invention, said infectious cDNA construct, said infectious viral particle or said pharmaceutical composition and combinations of the aforementioned may be administered either in a single dose, or in multiple doses to a subject in need thereof. The multiple doses may be administered concurrently, or consecutively (e.g., over a period of days or weeks). Typically, in therapeutic applications the treatment would be for the duration of the disease condition, e.g., at least until the respective cancer is no longer detectable by conventional means. Typical treatment regimes are known in the state of the art.

According to the present invention a “subject in need” may be any mammal in need of treatment according to the invention. The subject may be a human or an individual of any species of social, economic or research importance Including, but not limited to mice, rats, dogs, cats, sheep, goats, cows, horses, pigs, non-human primates, and humans. In a preferred embodiment of the present invention, the subject in need is a human.

The mode of administering to said subject in need thereof may be orally, intratumorally, peritumorally, intravenously, intraperitoneally and/or intramuscularly.

According to the present invention, said infectious cDNA construct, said infectious viral particle or said pharmaceutical composition and combinations of the aforementioned may be administered to a respective cancer in the individual subject in need thereof. A combination of different serotypes and/or different strains and/or different species and/or different genera of CVB3 virus, such as CVB3 virus from different species of animal, may also be alternatively used. If desired, the CVB3 can be chemically or biochemically pretreated, e.g., by treatment with a protease, such as chymotrypsin or trypsin prior to administration to the neoplasm. Such pretreatment will lead to removal of the outer coat of the virus and may thus result in improved infectivity of the virus. Combinations of at least two different viral particles as well as combinations of at least two different infectious cDNA constructs may also be administered. Also, combinations of the said at least two different infectious cDNA constructs with at least one viral particle may be administered. And, combinations of the said at least two different viral particles together with at least one infectious cDNA constructs may be administered. The infectious CVB3 cDNA construct and/or the CVB3 viral particle may be administered or applied in combination with other additional CVB or vector viruses, or additional infectious cDNA construct encoding different modified CVB viruses.

### REFERENCES

Andreoletti, L.; Leveque, N.; Boulagnon, C.; Brasselet, C.; Fornes, P. (2009): Viral causes of human myocarditis. In: Archives of cardiovascular diseases 102 (6-7), S. 559-568. DOI: 10.1016/j.acvd.2009.04.010.
Andtbacka, R. H.; Kaufman, H. L.; Collichio, F.; Amatruda, T.; Senzer, N.; Chesney, J. et al. (2015): Talimogene Laherparepvec Improves Durable Response Rate in Patients With Advanced Melanoma. In: Journal of clinical oncology: official journal oftheAmerican Society of Clinical Oncology33 (25), S. 2780-2788. DOI: 10.1200/JCO.2014.58.3377.
Donina, Simona; Strele, leva; Proboka, Guna; Auzins, Jurgis; Alberts, Peteris; Jonsson, Bjorn et al. (2015): Adapted ECHO-7 virus Rigvir immunotherapy (oncolytic virotherapy) prolongs survival in melanoma patients after surgical excision of the tumour in a retrospective study. In: Melanoma research 25 (5), S. 421-426. DOI: 10.1097/CMR.0000000000000180.
Fechner, H.; Pinkert, S.; Geisler, A.; Poller, W.; Kurreck, J. (2011): Pharmacological and biological antiviral therapeutics for cardiac coxsackievirus infections. In: Molecules 16 (10), S. 8475-8503. DOI: 10.3390/molecules16108475.
Fechner, H.; Sipo, I.; Westermann, D.; Pinkert, S.; Wang, X.; Suckau, L. et al. (2008): Cardiac-targeted RNA interference mediated by an AAV9 vector improves cardiac function in coxsackievirus B3 cardiomyopathy. In: Journal of molecular medicine 86 (9), S. 987-997. DOI: 10.1007/s00109-008-0363-x.
Garber, Ken (2006): China approves world's first oncolytic virus therapy for cancer treatment. In: Journal of the National Cancer Institute 98 (5), S. 298-300. DOI: 10.1093/jnci/djj111.
Hazini,Ahmet; Dieringer, Babette; Pryshliak, Markian; Knoch, Klaus-Peter; Heimann, Lisanne; Tolksdorf, Beatrice et al. (2021): miR-375- and miR-1-Regulated Coxsackievirus B3 Has No Pancreas and Heart Toxicity But Strong Antitumor Efficiency in Colorectal Carcinomas. In: Human Gene Therapy 32 (3-4), S. 216-230. DOI: 10.1089/hum.2020.228.
Hazini, Ahmet; Pryshliak, Markian; Bruckner, Vanessa; Klingel, Karin; Sauter, Martina; Pinkert, Sandra et al. (2018): Heparan Sulfate Binding Coxsackievirus B3 Strain PD: A NovelAvirulent Oncolytic Agent Against Human Colorectal Carcinoma. In: Human Gene Therapy 29 (11), S. 1301-1314. DOI: 10.1089/hum.2018.036.
He, F.; Yao, H.; Wang, J.; Xiao, Z.; Xin, L.; Liu, Z. et al. (2015): Coxsackievirus B3 engineered to contain microRNA targets for muscle-specific microRNAs displays attenuated cardiotropic virulence in mice. In: Journal of virology 89 (2), S. 908-916. DOI: 10.1128/JVI.02933-14.
Jia, Yang; Miyamoto, Shohei; Soda, Yasushi; Takishima, Yuto; Sagara, Miyako; Liao, Jiyuan et al. (2019): Extremely Low Organ Toxicity and Strong Antitumor Activity of miR-34-Regulated Oncolytic Coxsackievirus B3. In: Molecular Therapy—Oncolytics 12, S. 246-258. DOI: 10.1016/j.omto.2019.01.003.
Kelly, E. J.; Hadac, E. M.; Greiner, S.; Russell, S. J. (2008): Engineering microRNA responsiveness to decrease virus pathogenicity. In: Nature medicine 14 (11), S. 1278-1283. DOI: 10.1038/nm.1776.
Liu, Huitao; Xue, Yuan Chao; Deng, Haoyu; Mohamud, Yasir; Ng, Chen Seng; Chu, Axel et al. (2020): MicroRNA Modification of Coxsackievirus B3 Decreases Its Toxicity, while Retaining Oncolytic Potency against Lung Cancer. In: Molecular therapy oncolytics 16, S. 207-218. DOI: 10.1016/j.omto.2020.01.002.
Miyamoto, S.; Inoue, H.; Nakamura, T.; Yamada, M.; Sakamoto, C.; Urata, Y. et al. (2012): Coxsackievirus B3 is an oncolytic virus with immunostimulatory properties that is active against lung adenocarcinoma. In: Cancer research 72 (10), S. 2609-2621. DOI: 10.1158/0008-5472.CAN-11-3185.
Pinkert, Sandra; Pryshliak, Markian; Pappritz, Kathleen; Knoch, Klaus; Hazini, Ahmet; Dieringer, Babette et al. (2020): Development of a new mouse model for coxsackievirus-induced myocarditis by attenuating coxsackievirus B3 virulence in the pancreas. In: Cardiovascular research 116 (10), S. 1756-1766. DOI: 10.1093/cvr/cvz259.
Pryshliak, Markian; Hazini, Ahmet; Knoch, Klaus; Dieringer, Babette; Tolksdorf, Beatrice; Solimena, Michele et al. (2020): MiR-375-mediated suppression of engineered coxsackievirus B3 in pancreatic cells. In: FEBS letters 594 (4), S. 763-775. DOI: 10.1002/1873-3468.13647.
Scharfmann, Raphael; Pechberty, Severine; Hazhouz, Yasmine; Bulow, Manon von; Bricout-Neveu, Emilie; Grenier-Godard, Maud et al. (2014): Development of a conditionally immortalized human pancreatic p cell line. In: The Journal of clinical investigation 124 (5), S. 2087-2098. DOI: 10.1172/JC172674.
Spur, E. M.; Althof, N.; Respondek, D.; Klingel, K.; Heuser, A.; Overkleeft, H. S.; Voigt, A. (2016): Inhibition of chymotryptic-like standard proteasome activity exacerbates doxorubicin-induced cytotoxicity in primary cardiomyocytes. In: Toxicology 353-354, S. 34-47. DOI: 10.1016/j.tox.2016.04.010.
Wang, Chen; Fung, Gabriel; Deng, Haoyu; Jagdeo, Julienne; Mohamud, Yasir; Xue, Yuan Chao et al. (2019): NLRP3 deficiency exacerbates enterovirus infection in mice. In: FASEB journal: official publication of the Federation of American Societies for Experimental Biology 33 (1), S. 942-952. DOI: 10.1096/fj.201800301RRR.

### SHORT DESCRIPTION OF THE FIGURES

FIG. 1: miR-375 and miR-1 expression as well as miR-34a expression in colorectal cancer cells and structure of miR-TS viruses and their replication in HeLa cells.

A. Expression of miR-375 and miR-1 in colorectal carcinoma cells. Relative expression level of miR-375 and miR-1 in indicated colorectal carcinoma cell lines, HeLa cells, HEK293T cells, pancreatic EndoC-βH1 cells and embryonic mouse cardiomyocytes (EMCM), as well as in murine organs. Expression levels were determined by quantitative RT-PCR. Each miR expression level was normalized against level of endogenous U6 snRNA expression and is shown relative to miR-375 levels of the pancreas (left diagram) and miR-1 expression levels of the heart (right diagram) which were set to 1. The data represent the means±SEM of three independent experiments, each in triplicate.

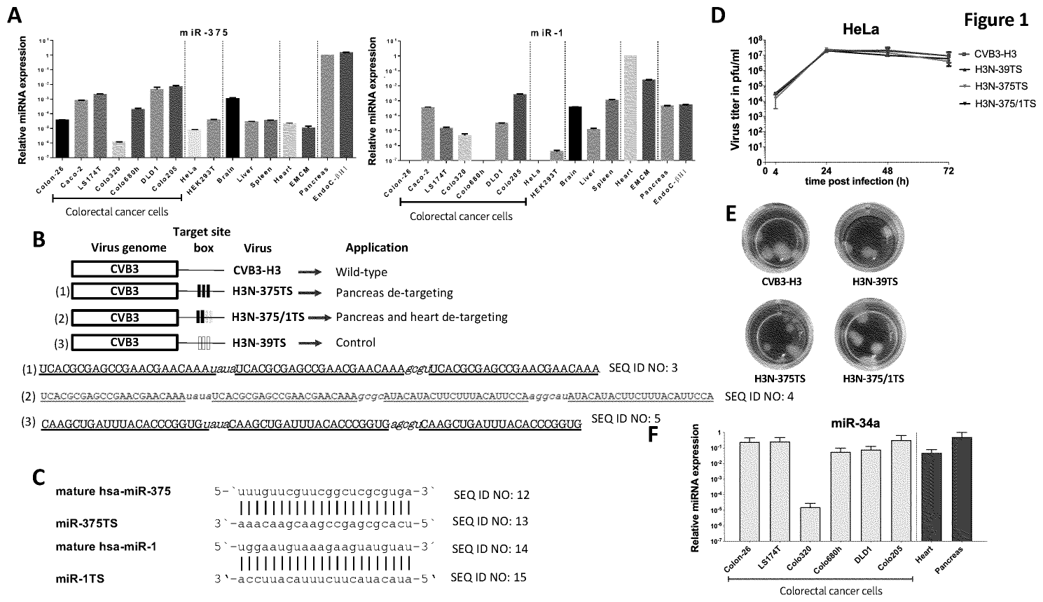

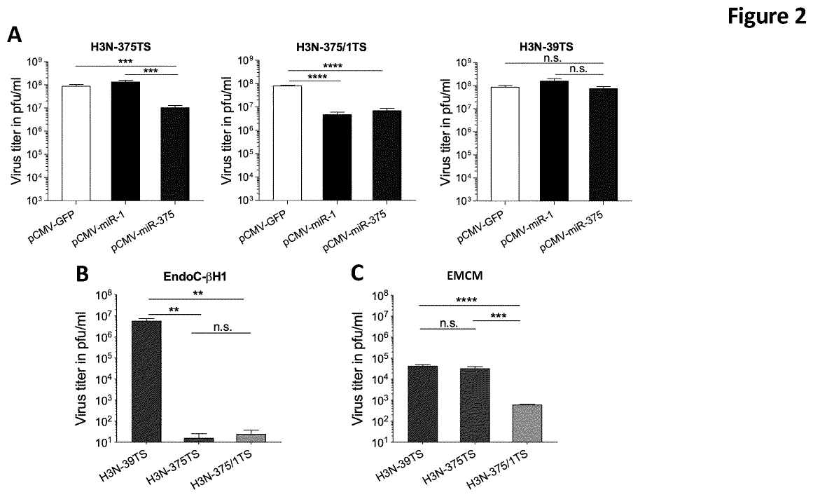

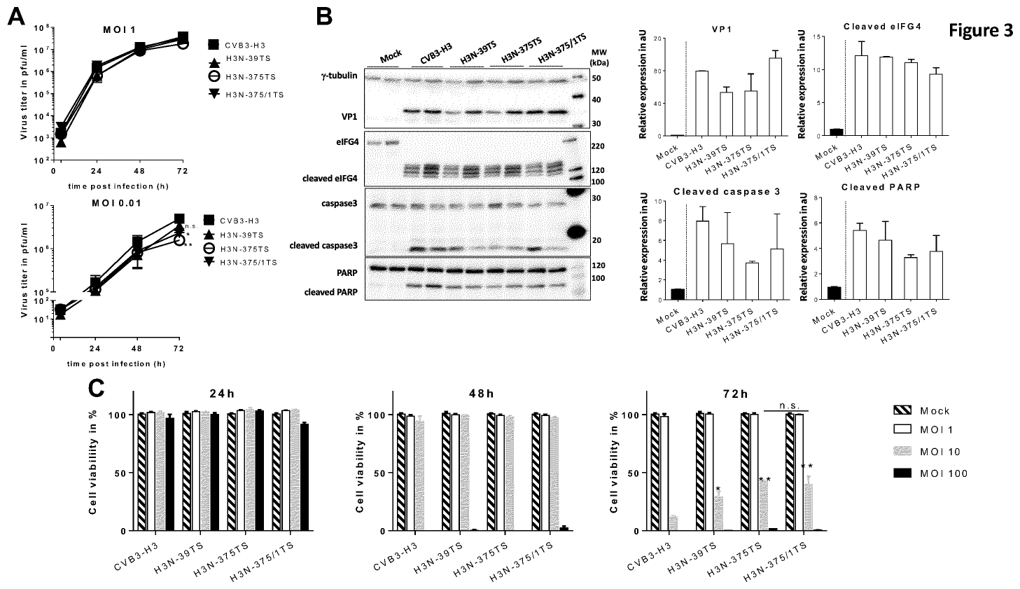

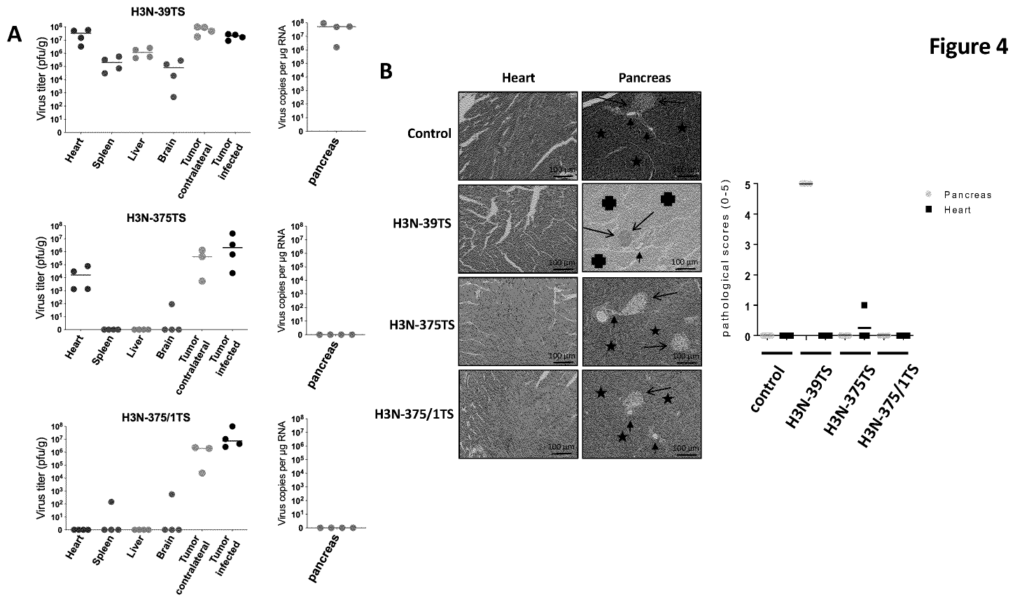

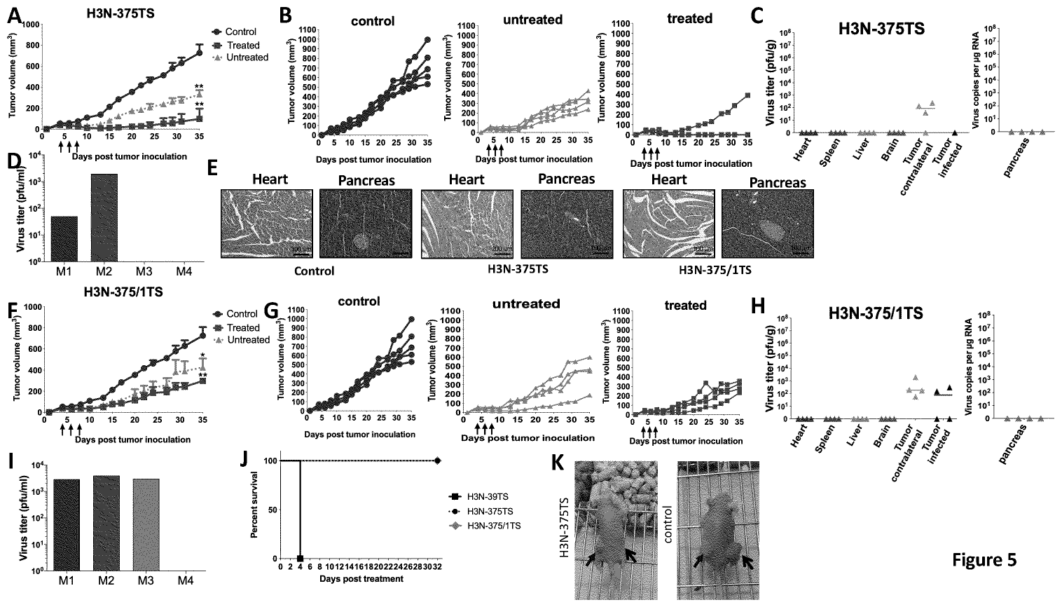

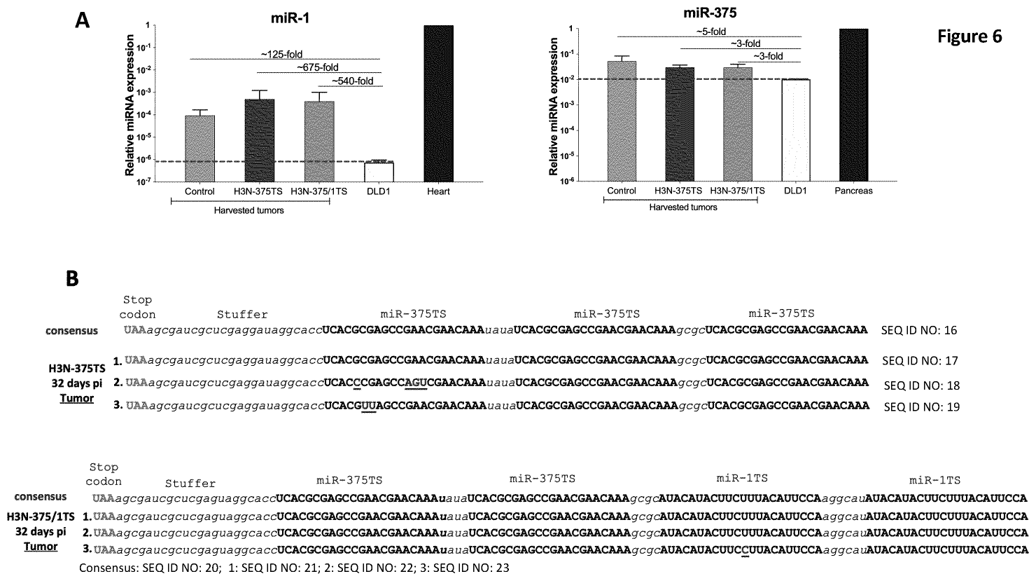

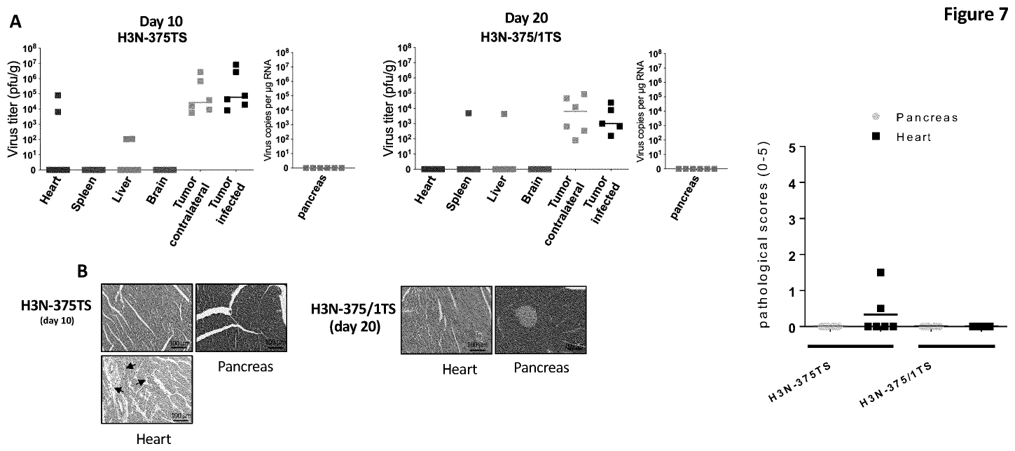

B. Structure of the inventive CVB3 variant H3, miR-TS bearing CVB3-H3 variants and miR-TS construct sequences (depicted in RNA code to show perfect base paring with respective mature cognate miR (cf. FIG. 1 C); in the infectious cDNA construct U (uracil) is replaced by T (thymine)). Upper panel: Illustration of target sites (TS) of miR-375 number (1 and 2) black), miR-1 (number (2) light grey) and miR-39 ((3) open bars)), virus names and virus application. The miR-TS sequences were inserted into the 3′ UTR region of virus genome of the CVB3 variant H3 immediately after the stop codon of the 3D polymerase. Three different miR-TS bearing CVB3 variants were produced; H3N-375TS containing three copies of target sites of the miR-375, H3N-375/1TS containing two copies of the target sites of miR-375 and two copies of the target sites of the miR-1 and the control virus H3N-39TS containing three copies of target sites of the miR-39, which is not expressed in mammalian cells. Lower panel: Sequences of respective miR-TS. Each miR-TS is underlined and is written in capital letters. Spacer sequences of four to eight nucleotides (shown in italics and small letters) were inserted between each miR-TS to improve miR-binding. Sequence of each miR-TS copy has 100% homolog to the full-length sequence of the cognate mature miR. Lower panel (nucleotide sequences): (1)—SEQ ID No: 3; (2)—SEQ ID No: 4; (3)—SEQ ID No: 5.

The nucleic acid sequence of the inventive infectious cDNA construct is defined by a nucleic acid sequence comprising:

5′-AAGCGATCGCTCGAGGATAGGCACCTCACGCGAGCCGAACGAACAAA
tataTCACGCGAGCCGAACGAACAAAgcgcTCACGCGAGCCGAACGAACA
AAAATGACCGTGGTTTAAA-3′ (SEQ ID No: 6, miR-375TS
construct)

or by a nucleic acid sequence comprising:

5′-TCACGCGAGCCGAACGAACAAAtataTCACGCGAGCCGAACGAACAA
AgcgcTCACGCGAGCCGAACGAACAAA-3′ (SEQ ID No: 7,
miR-375TS construct without stuffer sequence)

or by a nucleic acid sequence comprising:

5′-AGCGATCGCTCGAGGATAGGCACCTCACGCGAGCCGAACGAACAAAt
ataTCACGCGAGCCGAACGAACAAAgcgcATACATACTTCTTTACATTCC
AaggcctatATACATACTTCTTTACATTCCA-3′ (SEQ ID No: 8;
miR-375TS/miR-1TS construct)

or by a nucleic acid sequence comprising:

5′-TCACGCGAGCCGAACGAACAAAtataTCACGCGAGCCGAACGAACAA
AgcgcATACATACTTCTTTACATTCCAaggcctatATACATACTTCTTTA
CATTCCA-3′ (SEQ ID No: 9; miR-375TS/miR-1TS
construct without stuffer sequence)

Regarding SEQ ID No: 6, SEQ ID No: 7, SEQ ID No: 8, SEQ ID No: 9: If present, stuffer sequences have capital letters, which are written in italics. Each miR-TS is underlined and is written in capital letters. Spacer sequences of four to eight nucleotides (shown in italics and small letters). The last spacer sequence in SEQ ID No: 8 and SEQ ID No: 9 (aggcctat) can be also replaced by aggcat. The last spacer sequence in SEQ ID No: 6 and SEQ ID No: 7 (gcgc) can be also replaced by (gcgt).

The genomic sequence of a Coxsackievirus B3 (CVB3) of the inventive infectious cDNA construct is defined by a nucleic acid sequence of a CVB3 H3 comprising:

5′-TTAAAACAGCCTGTGGGTTGATCCCACCCACAGGGCCTATTGGGCGCTAGCACTCTGGTATCACGGTA
CCTTTGTGCGCCTGTTTTATATCCCCTCCCCCAACTGTAACTTAGAAGTAACACACTCCGATCAACAGTCAG
CGTGGCACACCAGCCATGTTTTGATCAAGCACTTCTGTTACCCCGGACTGAGTATCAATAGACTGCTCACG
CGGTTGAAGGAGAAAGCGTTCGTTATCCGGCCAACTACTTCGAAAAACCCAGTAACACCATAGAGGTTGCA
GAGTGTTTCGCTCAGCACTACCCCAGTGTAGACCAGGCCGATGAGTCACCGCATTCCCCACGGGCGACCG
TGGCGGTGGCTGCGTTGGCGGCCTGCCTATGGGGAAACCCATAGGACGCTCTAATACAGACATGGTGCGA
AGAGTCTATTGAGCTAGTTGGTAATCCTCCGGCCCCTGAATGCGGCTAATCCTAACTGCGGAGCACACACC
CTCAAACCAGAGGGCAGTGTGTCGTAACGGGCAACTCTGCAGCGGAACCGACTACTTTGGGTGTCCGTGT
TTCATTTTATTCCTATACTGGCTGCTTATGGTGACAATTGAGAGATTGTTACCATATAGCTATTGGATTGGC
CATCCGGTGTCTAATAGAGCTATTATATATCTCTTTGTTGGATTTATACCACTTAGCTTGAGAGAGGTTAAA
ACATTACAATTCATTGTTAAATTGAATACAACAAAATGGGAGCTCAAGTATCAACGCAAAAGACTGGGGCAC
ATGAGACCGGGCTGAATGCTAGCGGCAATTCCATTATTCACTACACGAATATTAATTATTACAAAGACGCCG
CATCCAACTCAGCCAATCGGCAGGATTTCACTCAAGACCCGGGCAAGTTCACAGAACCAGTGAAAGATATC
ATGATTAAATCACTACCAGCTCTCAACTCCCCCACAGTAGAGGAGTGCGGATACAGTGACAGGGTGAGATC
AATCACACTAGGTAACTCCACCATAACGACTCAGGAATGCGCTAACGTGGTGGTAGGCTATGGAGTGTGGC
CAGATTATCTGAAGGATAGCGAGGCTACAGCAGAGGACCAACCGACCCAACCAGACGTTGCCACATGTAGG
TTCTATACCCTTGACTCTGTACAATGGCAGAAAACCTCACCAGGATGGTGGTGGAAGCTGCCTGATGCTTT
GTCGAACTTAGGACTGTTTGGGCAGAACATGCAGTACCACTACTTGGGCCGAACTGGGTATACCATACATG
TGCAGTGCAATGCATCCAAGTTCCACCAAGGATGCTTGCTAGTAGTGTGTGTACCGGAAGCTGAGATGGGT
TGCGCAACGCTAAACAACACCCCATCCAGTGCAGAATTGCTGGGGGGCGATAGCGCCAAAGAGTTTGCGG
ACAAACCGGTTGCATCCGGGTCCAACAAGTTGGTACAGAGGGTGGTGTATAATGCAGGCATGGGGGTGGG
TGTTGGAAACCTTACCATTTTCCCTCACCAGTGGATCAATCTACGCACCAACAATAGTGCTACAATTGTGAT
GCCATACACCAACAGCGTACCTATGGATAACATGTTTAGGCATAACAACGTCACCCTAATGGTTATCCCATT
TGTACCGCTAGATTACTGCCCTGGGTCTACCACGTACGTCCCAATCACGATCACGATAGCCCCAATGTGTG
CCGAGTACAATGGACTACGTTTGGCCGGGCACCAGGGCTTACCAACCATGAACACTCCGGGGAGCTGTCA
ATTTCTGACATCAGACGACTTCCAATCACCATCTGCCATGCCGCAATACGACGTCACGCCAGAGATGAGGA
TACCTGGTGAGGTGAAGAACTTGATGGAAATAGCTGAGGTTGACTCAGTTGTCCCGGTCCAAAATGTTGGA
GAGAAGGTCAACTCCATGGAAGCGTACCAGATACCTGTGAGATCCAATGAAGGATCTGGAACGCAAGTATT
CGGCTTCCCACTGCAACCAGGGTATTCGAGTGTTTTCAGTCGGACGCTCCTAGGAGAGATCTTGAACTATT
ACACCCATTGGTCAGGCAGCATAAAGCTTACGTTTATGTTCTGTGGTTCGGCCATGGCCACTGGAAAATTC
CTTTTGGCATACTCACCACCAGGCGCTGGGGCTCCCACAAAAAGGGTTGATGCTATGCTTGGCACTCATGT
AGTTTGGGATGTGGGGCTACAATCAAGTTGCGTGCTGTGCATACCCTGGATAAGCCAAACACACTACCGGT
ATGTTGCTTCAGATGAGTATACCGCAGGGGGTTTTATTACGTGCTGGTATCAAACAAACATAGTCGTCCCA
GCAGATGCCCAAAGCTCCTGTTACATCATGTGTTTCGTATCAGCATGCAATGATTTCTCTGTCAGGCTATT
GAAGGACACTCCTTTTATTTCGCAGCAAAACTTTTTCCAGGGCCCCGTGGAAGACGCGATAACAGCCGCCA
TAGGGAGAGTTGCGGACACCGTGGGTACAGGGCCAACCAACTCAGAGGCTATACCAGCACTCACTGCTGC
TGAGACAGGTCACACGTCGCAAGTAGTGCCGAGTGACACCATGCAGACACGCCACGTTAAGAACTACCATT
CAAGGTCTGAGTCGACCATAGAGAACTTCCTATGTAGGTCAGCATGCGTGTACTTTACAGAGTATGAAAAC
TCAGGCGCCAAGCGGTATGCTGAATGGGTAATAACACCACGACAAGCGGCACAACTTAGGAGAAAGCTAGA
ATTCTTTACCTACGTCCGGTTTGACCTGGAGCTGACGTTTGTCATAACAAGTACTCAACAGCCCTCAACCAC
ACAGAACCAGGATGCACAGATCCTAACACACCAAATTATGTATGTACCACCAGGTGGGCCTGTGCCAGACA
AAGTCGATTCTTACGTGTGGCAAACATCTACGAATCCCAGTGTGTTTTGGACCGAGGGAAACGCCCCGCCG
CGTATGTCCGTACCGTTTTTGAGCATTGGCAACGCTTATTCAAATTTCTATGATGGGTGGTCTGAATTTTC
CAGGAACGGGGTTTACGGTATCAACACACTAAACAACATGGGCACGCTATATGCAAGACATGTCAATGCTG
GAAGCACGGGACCAATAAAAAGCACCATTAGAATCTACTTCAAACCTAAGCATGTCAAAGCGTGGATACCTA
GACCACCTAGACTCTGCCAATACGAGAAGGCAAAGAACGTGAACTTCCAACCCAGCGGAGTTACCACTACT
AGGCAAAGCATCACTACAATGACAAATACGGGCGCATTTGGACAACAATCAGGGGCAGTATACGTAGGGAA
CTACAGGGTAGTAAATAGACATCTAGCTACCAGTGCTGACTGGCAAAACTGTGTGTGGGAAAATTACAACA
GAGACCTCTTAGTGAGCACGACCACAGCACATGGATGTGATATTATAGCCAGATGTCGGTGTACAACGGGA
GTGTACTTTTGTGCGTCCAAAAACAAACACTACCCAATTTCATTTGAAGGACCAGGTATAGTAGAGGTCCAA
GAGAGTGAGTACTACCCTAGGAGATACCAATCCCATGTGCTTTTAGCAGCTGGGTTTTCCGAACCAGGTGA
CTGTGGCGGTATCCTAAGGTGTGAGCATGGTGTCATTGGCATTGTGACCATGGGGGGTGAAGGCGTGGTC
GGCTTTGCAGACATCCGTGATCTCCTGTGGCTGGAAGATGATGCAATGGAACAGGGAGTGAAGGACTATG
TGGAACAGCTTGGAAATGCATTCGGCTCTGGCTTCACTAACCAAATATGTGAGCAAGTCAACCTCCTGAAA
GAATCACTAGTGGGTCAAGACTCCATCTTAGAGAAGTCTCTAAAAGCCTTAGTTAAGATAATATCAGCCTTA
GTAATTGTGGTGAGGAACCACGATGACCTAATCACGGTGACTGCCACACTAGCCCTCATTGGTTGTACCTC
GTCCCCATGGCGGTGGCTTAAGCAGAAAGTGTCCCAATATTACGGAATACCCATGGCTGAACGCCAAAACA
ACGGATGGCTAAAAAAGTTCACTGAGATGACAAACGCCTGCAAGGGCATGGAATGGATAGCCATTAAGATT
CAGAAATTCATTGAGTGGCTCAAAGTTAAAATTTTACCTGAGGTCAGGGAAAAACACGAATTCCTGAACAGA
CTCAAACAGCTCCCCCTGTTAGAGAGTCAAATTGCCACAATCGAGCAAAGTGCACCGTCACAGAGTGACCA
GGAGCAATTGTTTTCCAATGTCCAATACTTTGCTCACTATTGCAGAAAGTATGCTCCCCTTTATGCATCAGA
GGCAAAGAGAGTGTTCTCCCTTGAGAAGAAGATGAGTAATTACATACAGTTCAAGTCCAAATGCCGTATTG
AGCCTGTATGTCTGCTCCTGCATGGGAGTCCCGGTGCAGGTAAGTCAGTTGCAACAAATCTGATCGGAAGA
TCACTCGCGGAAAAGTTAAACAGCTCAGTGTATTCACTACCCCCAGACCCAGATCACTTCGATGGCTATAA
ACAGCAGGCTGTAGTGATCATGGACGATCTATGTCAGAACCCCGATGGGAAAGATGTCTCCTTGTTCTGTC
AGATGGTTTCCAGTGTGGATTTTGTACCACCCATGGCCGCCCTGGAAGAGAAAGGCATCTTGTTCACCTCC
CCGTTCGTTTTGGCATCAACCAATGCGGGATCTATTAACGCTCCAACTGTGTCAGACAGCAGGGCCTTAGC
AAGGAGATTCCACTTTGACATGAATATTGAAGTTATTTCTATGTACAGCCAAAATGGCAAGATAAACATGCC
AATGTCAGTGAAGACGTGTGATGAAGAGTGTTGCCCAGTCAACTTTAAGAAATGTTGCCCGTTAGTCTGTG
GAAAGGCCATCCAATTCATAGACAGAAGAACTCAAGTCAGATACTCCCTCGATATGCTGGTAACTGAGATG
TTTAGGGAATACAACCACAGGCACAGTGTCGGGGCTACCCTTGAGGCACTGTTCCAGGGTCCACCAGTATA
CAGAGAGATTAAGATTAGCGTGGCACCAGAAACACCACCACCACCAGCTATCGCGGACTTGCTTAAATCAG
TGGATAGCGAAGCCGTGAGAGAGTATTGCAAAGAAAAGGGATGGTTGGTTCCTGAGGTCAACTCCACCCT
CCAAATTGAAAAACATGTCAGTCGGGCTTTCATCTGCTTGCAGGCAATAACTACGTTTGTGTCAGTAGCTG
GAATCATCTATATAATATACAAGCTCTTTGCAGGCTTTCAAGGTGCATATACAGGAATACCCAACCAGAAGC
CCAAGGTACCTACCCTAAGGCAAGCAAAAGTGCAGGGTCCTGCATTTGAATTTGCTGTTGCAATGATGAAG
AGGAACTCAAGCACAGTGAAGACAGAGTATGGTGAGTTCACCATGTTGGGCATTTATGATAGGTGGGCCGT
TTTGCCACGTCATGCCAAACCCGGACCAACCATCCTGATGAATGACCAGGAGGTAGGCGTGCTGGACGCTA
AAGAGTTAGTGGATAAGGATGGTACAAACCTAGAACTGACACTGCTCAAGTTGAACAGGAACGAGAAGTTC
AGAGACATCAGAGGCTTCTTAGCAAAGGAGGAGGTGGAAGTCAACGAGGCCGTGCTAGCAATTAATACCA
GTAAATTTCCCAACATGTACATTCCGGTGGGACAAGTCACGGATTACGGTTTCCTAAACCTGGGTGGTACG
CCCACTAAAAGAATGCTTATGTACAACTTCCCCACGAGAGCAGGTCAATGTGGCGGAGTACTCATGTCCAC
CGGCAAAGTCCTGGGGATCCATGTTGGTGGAAATGGTCATCAAGGTTTCTCAGCGGCACTTCTCAAGCACT
ATTTCAATGATGAACAAGGAGAGATCGAGTTTATTGAGAGCTCAAAGGAAGCAGGGTTCCCTATTATCAAC
ACACCTAGTAAGACTAAGCTGGAGCCGAGTGTCTTCCACCAGGTTTTTGAAGGTGACAAAGAGCCAGCGGT
CCTCAGGAATGGTGATCCACGCCTCAAAGTCAACTTTGAGGAGGCCATATTTTCCAAGTACATCGGGAATG
TTAACACACACGTGGATGAATACATGATGGAGGCTGTTGACCATTATGCCGGACAATTGGCCACCCTAGAC
ATTAGCACTGAACCAATGAAGTTGGAGGATGCTGTATACGGTACTGAAGGCCTTGAGGCTCTTGATCTAAC
AACGAGTGCAGGTTACCCTTATGTCGCCCTGGGCATCAAGAAGAGAGACATCCTCTCAAAGAAGACCAGGG
ACCTTACTAAGCTGAAAGAGTGCATGGATAAGTACGGTCTAAACCTACCAATGGTAACCTATGTGAAAGAC
GAACTCAGATCTGCAGAGAAGGTGGCAAAGGGAAAGTCCAGGCTCATTGAGGCGTCCAGTTTGAATGACT
CTGTGGCAATGAGACAGACATTCGGCAACTTGTACAAAACTTTTCACCTAAACCCAGGGATTGTGACTGGC
AGTGCTGTCGGGTGTGACCCGGACCTCTTTTGGAGTAAAATACCAGTGATGTTGGACGGTCATCTCATAGC
TTTTGATTATTCTGGATATGATGCTAGCTTGAGTCCCGTATGGTTTGCTTGTTTAAAACTACTACTTGAAAA
ACTTGGTTACTCGCACAAGGAGACCAATTACATTGATTACCTGTGCAACTCCCATCACCTGTACAGGGACA
AACATTATTTTGTGCGGGGTGGCATGCCATCTGGATGTTCTGGCACAAGCATCTTTAACTCAATGATAAAT
AACATCATAATCAGGACACTCATGCTGAAGGTGTACAAAGGGATCGACTTGGATCAATTCAGGATGATTGC
TTATGGTGACGATGTGATTGCATCATACCCGTGGCCCATAGATGCGTCTTTGCTTGCTGAAGCTGGCAAGG
ACTATGGATTAATCATGACACCAGCAGACAAAGGGGAGTGCTTCAATGAAGTTACTTGGACTAACGTCACA
TTCCTAAAGAGGTATTTTAGAGCAGATGAACAATACCCCTTTTTAGTGCACCCCGTTATGCCCATGAAAGAC
ATACACGAATCAATCAGATGGACCAAGGATCCAAAGAATACCCAAGACCATGTGCGCTCATTGTGCTTATT
GGCCTGGCACAACGGGGAGCACGAATATGAGGAGTTTATCCGCAAAATCAGGAGCGTCCCAGTTGGACGT
TGTTTGACTCTACCTGCGTTCTCAACCTTACGTAGGAAGTGGTTGGACTCTTTCTAAATTAGAGACAATTTG
ATCTGATTTGAATTGGCTTAACCCTACTGTACTAACCGAACTAGACAACGGTGCAGTAGGGGTAAATTCTC
CGCATTCGGTGCGGAAAAAAAAAAAAAAAAA -3′ (SEQ ID No: 10; CVB3 H3).

Alternatively the genomic sequence of a Coxsackievirus B3 (CVB3) of the inventive infectious cDNA construct is defined by a nucleic acid sequence of a CVB3 PD-0, e.g. CVB3 rPD comprising:

5′- TTAAAACAGCCTGTGGGTTGATCCCACCCACAGGGCCCATTGGGCGCTAGCACTCTGGTATCACGGT
ACCTTTGTGCGCCTGTTTTATACCCCCCCCCCCAACTGTAACTTAGAAGCAACACACACCGATCAACAGTCA
GCGTGGCACACCAGCCACGTTTTGATCAAGCACTTCTGTTACCCCGGACTGAGTATCAATAGACTGCTCAC
GCGGTTGAAGGAGAAAGCGTTCGTTATCCGGCCAACTATTTCGAAAAACCTAGTAACACCGTGGAAGTTGC
AGAGTGTTTCGCTCAGCACTACCCCAGTGTAGATCAGGTCGATGAGTCACCGCATTCCCCACGGGCGACC
GTGGCGGTGGCTGCGTTGGCGGCCTGCCCATGGGGAAACCCATGGGACGCTCTAATACAGACATGGTGCG
AAGAGTCTATTGAGCTAGTTGGTAGTCCTCCGGCCCCTGAATGCGGCTAATCCTAACTGCGGAGCACACAC
CCTCAAGCCAGAGGGCAGTGTGTCGTAACGGGCAACTCTGCAGCGGAACCGACTACTTTGGGTGTCCGTG
TTTCATTTTATTCCTATACTGGCTGCTTATGGTGACAATTGAGAGATTGTTACCATATAGCTATTGGATTGG
CCATCCGGTGACCAATAGAGCTATTATATATCTCTTTGTTGGGTTTATACCACTTAGCTTGAAAGAGGTTAA
AACATTACAATTCATTGTTAAGTTGAATACAGCAAAATGGGAGCTCAAGTATCAACGCAAAAGACTGGGGCA
CATGAGACCGGGCTGAATGCTAGCGGCAATTCCATCATTCACTACACAAATATTAATTATTACAAGGATGCC
GCATCCAACTCAGCCAATCGGCAGGATTTCGCTCAAGACCCGGGCAAGTTCACAGAACCAGTAAAAGATAT
CATGATTAAATCACTACCAGCTCTCAACTCCCCCACAGTAGAGGAGTGCGGATACAGTGACAGGGTGAGAT
CAATCACATTAGGTAACTCCACCATAACGACTCAGGAATGCGCCAACGTGGTGGTGGGCTATGGAGTATGG
CCAGATTATCTAAAGGATAGTGAGGCAACAGCAGAGGACCAACCGACCCAACCAGACGTTGCCACATGTAG
GTTCTATACCCTTGACTCTGTGCAATGGCAGAAAACCTCACCAGGATGGTGGTGGAAGCTGCCCGATGCTT
TGTCGAACTTAGGACTGTTTGGGCAGAACATGCAGTACCACTACTTAGGCCGAACTGGGTATACCGTACAT
GTGCAGTGCAATGCATCTAAGTTCCACCAAGGATGCTTGCTAGTAGTGTGTGTACCGGAAGCTGAGATGG
GTTGCGCAACGCTAGACAACACCCCATCCAGTGCAGAATTGCTGGGGGGCGATAGCGCAAAAGAGTTTGC
GGACAAACCGGTCGCATCCGGGTCCAACAAGTTGGTACAGAGGGTGGTGTATAATGCAGGCATGGGGGTG
GGTGTTGGAAACCTCACCATTTTCCCCCACCAATGGATCAACCTACGCACCAATAATAGTGCTACAATTGTG
ATGCCATACACCAACAGTGTACCTATGGATAACATGTTTAGGCATAACAACGTCACCCTAATGGTTATCCCA
TTTGTACCGCTAGATTACTGCCCTGGGTCCACCACGTACGTCCCAATTACGGTCACGATAGCCCCAATGTG
TGCCGAGTACAATGGGTTACGTTTAGCAGGGCACCAGGGCTTACCAACCATGAATACTCCGGGGAGCTGT
CAATTTCTGACATCAGACGACTTCCAATCGCCATCCGCCATGCCGCAATATGACGTCACACCAGAGATGAG
GATACCTGGTGAGGTGAAAAACTTGATGGAAATAGCTGAGGTTGACTCAGTTGTCCCAGTCCAAAATGTTG
GAGAGAAGGTCAACTCTATGGAAGCATACCAGATACCTGTGAGATCCAATGAAGGATCTGGAACGCAAGTA
TTCGGCTTTCCACTGCAACCAGGGTACTCGAGTGTTTTTAGTCGGACGCTCCTAGGAGAGATCTTGAACTA
TTATACACATTGGTCAGGCAGCATAAAGCTTACGTTTATGTTCTGTGGTTCGGCCATGGCTACTGGAAAAT
TCCTTTTGGCATACTCACCACCAGGTGCTGGAGCTCCTACAAAAAGGGTTGATGCCATGCTTGGTACTCAT
GTAGTTTGGGACGTGGGGCTACAATCAAGTTGCGTGCTGTGTATACCCTGGATAAGCCAAACACACTACCG
GTATGTTGCTTCAGATGAGTATACCGCAGGGGGTTTTATTACGTGCTGGTATCAAACAAACATAGTGGTCC
CAGCGGATGCCCAAAGCTCCTGTTACATCATGTGTTTTGTGTCAGCATGCAATGACTTCTCTGTCAGGCTA
TTGAAGGATACTCCTTTCATTTCGCAGCAAAACTTTTACCAGGGCCCAGTGGAAGACGCGATAACAGCCGC
TATAGGGAGAGTTGCGGATACCGTGGGTACAGGGCCAACCAACTCAGAAGCTATACCAGCACTCACTGCTG
CTGAGACAGGTCACACGTCACAAGTAGTGCCGGGTGACACCATGCAGACACGCCACGTTAAGAACTACCAT
TCAAGGTCCGAGTCAACCATAGAGAACTTCCTATGTAGGTCAGCATGCGTGTACTTTACGAAGTATGCAAA
CTCAGGTGCCAAGCGGTATGCTGAATGGGCAATAACACCACGACAAGCAGCACAACTTAGGAGAAAGCTAG
AATTCTTTACCTACGTCCGGTTCGACCTGGAGCTGACGTTTGTCATAACAAGTACTCAACAGCCCTCAACC
ACACAGAACCAAGACGCACAGATCCTAACACACCAAATTATGTATGTACCACCAGGTGGACCTGTACCAGA
GAAAGTTGATTCATACGTGTGGCAAACATCTACGAATCCCAGTGTGTTTTGGACCGAGGGAAACGCCCCGC
CGCGCATGTCCATACCGTTTTTGAGCATTGGCAACGCCTATTCAAATTTCTATGACGGATGGTCTGAATTT
TCCAGGAACGGAGTTTACGGCATCAACACGCTAAACAACATGGGCACGCTATATGCAAGACATGTCAACTC
TGGAAGCACGGGTCCAATAAAAAGCACCATTAGAATCTACTTCAAACCGAAGCATGTCAAAGCGTGGATAC
CTAGACCACCTAGACTCTGCCAATACGAGAAGGCAAAGAACGTGAACTTCCAACCCAGCGGAGTTACCACT
ACTAGGCAAAGCATCACTACAATGACAAATACGGGCGCATTTGGACAACAATCAGGGGCAGTGTATGTGGG
GAACTACAGGGTAGTAAATAGACATCTAGCTACCAGTGCTGACTGGCAAAACTGTGTGTGGGAAAGTTACA
ACAGAGACCTCTTAGTGAGCACGACCACAGCACATGGATGTGATATTATAGCCAGATGTCAGTGCACAACG
GGAGTGTACTTTTGTGCGTCCAAAAACAAGCACTACCCAATTTCGTTTGAAGGACCAGGTCTAGTAGAGGT
CCAAGAGAGTGAATACTACCCCAGGAGATACCAATCCCATGTGCTTTTAGCAGCTGGATTTTCCGAACCAG
GTGACTGTGGCGGTATCCTAAGGTGTGAGCATGGTGTCATTGGCATTGTGACCATGGGGGGTGAAGGCGT
GGTCGGCTTTGCAGACATCCGTGATCTCCTGTGGCTGGAAGATGATGCAATGGAACAGGGAGTGAAGGAC
TATGTGGAACAGCTTGGAAATGCATTCGGCTCCGGCTTTACTAACCAAATATGTGAGCAAGTCAACCTCCT
GAAAGAATCACTAGTGGGTCAAGACTCCATCTTAGAGAAATCTCTAAAAGCCTTAGTTAAGATAATATCAGC
CTTAGTAATTGTGGTGAGGAACCACGATGACCTGATCACTGTGACTGCCACACTAGCCCTTATCGGTTGTA
CCTCGTCCCCGTGGCGGTGGCTCAAACAGAAGGTGTCACAATATTACGGAATCCCTATGGCTGAACGCCAA
AACAATAGCTGGCTTAAGAAATTTACTGAAATGACGAATGCTTGCAAGGGTATGGAATGGATAGCTGTCAA
AATTCAGAAATTCATTGAATGGCTCAAAGTAAAAATTTTGCCAGAGGTCAGGGAAAAACACGAATTCCTGAA
CAGACTTAAACAACTCCCCTTATTAGAAAGTCAGATCGCCACAATCGAGCAGAGCGCGCCATCCCAAAGTG
ACCAGGAACAATTATTTTCCAATGTCCAATACTTTGCCCACTATTGCAGAAAGTACGCTCCCCTCTATGCAG
CTGAAGCAAAGAGGGTGTTCTCCCTTGAGAAGAAGATGAGCAATTACATACAGTTCAAGTCCAAATGCCGT
ATTGAACCTGTATGTTTGCTCCTGCACGGGAGCCCTGGTGCCGGCAAGTCGGTGGCAACAAACTTAATTGG
AAGGTCGCTTGCTGAGAAACTCAACAGCTCAGTGTACTCACTACCGCCAGACCCAGATCACTTCGACGGAT
ACAAACAGCAGGCCGTGGTGATTATGGACGATCTATGCCAGAATCCTGATGGGAAAGACGTCTCCTTGTTC
TGCCAAATGGTTTCCAGTGTAGATTTTGTACCACCCATGGCTGCCCTAGAAGAGAAAGGCATTCTGTTCAC
CTCACCGTTTGTCTTGGCATCGACCAATGCAGGATCTATTAATGCTCCAACCGTGTCAGATAGCAGAGCCT
TGGCAAGGAGATTTCACTTTGACATGAACATCGAGGTTATTTCCATGTACAGTCAGAATGGCAAGATAAAC
ATGCCCATGTCAGTCAAGACTTGTGACGATGAGTGTTGCCCGGTCAATTTTAGAAAGTGCTGCCCTCTTGT
GTGTGGGAAGGCTATACAATTCATTGATAGAAGAACACAGGTCAGATACTCTCTAGACATGCTAGTCACCG
AGATGTTTAGGGAGTACAATCATAGACATAGCGTGGGGACCACGCTTGAGGCACTGTTCCAGGGACCACCA
GTATACAGAGAGATCAAAATTAGCGTTGCACCAGAGACACCACCACCGCCCGCCATTGCGGACCTGCTCAA
ATCGGTAGACAGTGAGGCTGTGAGGGAGTACTGCAAAGAAAAAGGATGGTTGGTTCCTGAGATCAACTCC
ACCCTCCAAATTGAGAAACATGTCAGTCGGGCTTTCATTTGCTTACAGGCATTGACCACATTTGTGTCAGT
GGCTGGAATCATATATATAATATATAAGCTCTTTGCGGGTTTTCAAGGTGCTTATACAGGAGTGCCCAACC
AGAAGCCCAGAGTGCCTACCCTGAGGCAAGCAAAAGTGCAAGGCCCTGCCTTTGAGTTCGCCGTCGCAAT
GATGAAAAGGAACTCAAGCACGGTGAAAACTGAATATGGCGAGTTTACCATGCTGGGCATCTATGACAGGT
GGGCCGTTTTGCCACGCCACGCCAAACCTGGGCCAACCATCTTGATGAATGATCAAGAGGTTGGTGTGCTA
GATGCCAAGGAGCTAGTAGACAAGGACGGCACCAACTTAGAACTGACACTACTCAAATTGAACCGGAATGA
GAAGTTCAGAGACATCAGAGGCTTCCTAGCCAAGGAGGAAGTGGAGGTTAATGAGGCAGTGCTAGCAATT
AACACCAGCAAGTTTCCCAACATGTACATTCCAGTAGGACAGGTCACAGAATACGGCTTCCTAAACCTAGG
TGGCACACCCACCAAGAGAATGCTTATGTACAACTTCCCCACAAGAGCAGGCCAGTGTGGTGGAGTGCTCA
TGTCCACCGGCAAGGTACTGGGTATCCATGTTGGTGGAAATGGCCATCAGGGCTTCTCAGCAGCACTCCT
CAAACACTACTTCAATGATGAGCAAGGTGAAATAGAATTTATTGAGAGCTCAAAGGACGCCGGGTTTCCAG
TCATCAACACACCAAGTAAAACAAAGTTGGAGCCTAGTGTTTTCCACCAGGTCTTTGAGGGGAACAAAGAA
CCAGCAGTACTCAGGAGTGGGGATCCACGTCTCAAGGCCAATTTTGAAGAGGCTATATTTTCCAAGTATAT
AGGAAATGTCAACACACACGTGGATGAGTACATGCTGGAAGCAGTGGACCACTACGCAGGCCAACTAGCCA
CCCTAGATATCAGCACTGAACCAATGAAACTGGAGGACGCAGTGTACGGTACCGAGGGTCTTGAGGCGCT
TGATCTAACAACGAGTGCTGGTTACCCATATGTTGCACTGGGTATCAAGAAGAGGGACATCCTCTCTAAGA
AGACTAAGGACCTAACAAAGTTAAAGGAATGTATGGACAAGTACGGCCTGAACCTACCAATGGTGACTTAT
GTAAAAGATGAGCTCAGGTCCATAGAGAAGGTAGCGAAAGGAAAGTCTAGGCTGATTGAGGCGTCCAGTTT
GAATGATTCAGTGGCGATGAGACAGACATTTGGTAATCTGTACAAAACTTTCCACCTAAACCCAGGGGTTG
TGACTGGTAGTGCTGTTGGGTGTGACCCAGACCTCTTTTGGAGCAAGATACCAGTGATGTTAAATGGACAT
CTCATAGCATTTGATTACTCTGGGTACGATGCTAGCTTAAGCCCTGTCTGGTTTGCTTGCCTAAAAATGTTA
CTTGAGAAGCTTGGATACACGCACAAAGAGACAAACTACATTGACTACTTGTGTAACTCCCATCACCTGTAC
AGGGATAAACATTACTTTGTGAGGGGTGGCATGCCCTCGGGATGTTCTGGTACCAGTATTTTCAACTCAAT
GATTAACAACATCATAATTAGGACACTAATGCTAAAAGTGTACAAAGGGATTGACTTGGACCAATTCAGGAT
GATCGCATATGGTGATGATGTGATCGCATCGTACCCATGGCCTATAGATGCATCTTTACTCGCTGAAGCTG
GTAAGGGTTACGGGCTGATCATGACACCAGCAGATAAGGGAGAGTGCTTTAACGAAGTTACCTGGACCAAC
GTCACTTTCCTAAAGAGGTATTTTAGAGCAGATGAACAGTACCCCTTCCTGGTGCATCCTGTTATGCCCAT
GAAAGACATACACGAATCAATTAGATGGACCAAGGATCCAAAGAACACCCAAGATCACGTGCGCTCACTGT
GTTTATTGGCTTGGCATAACGGGGAGCACGAATATGAGGAGTTCATCCGTAAAATTAGAAGCGTCCCAGTC
GGACGTTGTTTGACCCTCCCCGCGTTTTCAACTCTACGCAGGAAGTGGTTGGACTCCTTTTAGATTAGAGA
CAATTTGAAATAATTTAGATTGGCTTAACCCTACTGTGCTAACCGAACCAGATAACGGTACAGTAGGGGTAA
ATTCTCCGCATTCGGTGCGGAAAAAAAAAAAAAAA -3′ (SEQ ID No: 11; CVB3 rPD).

C. Sequence comparison between miR-375 and miR-375TS and between miR-1 and miR-1TS.

Nucleotide sequences: mature hsa-miR-375-SEQ ID No: 12; miR-375TS—SEQ ID No: 13; mature hsa-miR-1-SEQ ID No: 14; miR-1TS—SEQ ID No: 15.

D. Replication kinetics of miR-TS equipped viruses in HeLa cells. HeLa cells were infected at an MOI of 0.1 of the respective viruses and plaque assays were performed on cell lysates collected at 4, 24, 48 and 72 h post infection (p.i.) to determine the titer of the viruses. The data represent the means±SEM of three independent experiments, each in triplicate.

E. Plaque morphology of miR-TS viruses and parental CVB3-H3 variant in HeLa cells. All viruses showed show similar plaque sizes.

F. Expression levels of miR-34a. Relative expression level of miR-34a in various human colorectal carcinoma cell lines (Colon-26, Caco-2, LS174T, Colo320, Colo680h, DLD1, Colo205), murine pancreas and heart. The quantification was determined by qRT-PCR. Each miR expression level was normalized against the expression level of U6 RNA in the same sample. The miR expression levels of the pancreas was set at 1. Note: compared to miR-1 expression levels in the heart and miR-375 expression levels in the pancreas, the expression of miR-34a was about 100-fold and 30-fold lower in both organs, respectively (results not shown).

FIG. 2: Inhibition of H3N-375TS and H3N-375/1 Ts by miR-375 and miR-1 in vitro

A. Inhibition of miR-TS replication in HEK-293T cells transiently transfected with miR-375 or miR-1. HEK293T cells were transfected with miR-1 or miR-375 expression plasmids or a control plasmid expressing GFP. Cells were infected 24 h later with H3N-375/1TS, H3N-375TS or H3N-39TS at an MOI of 0.1. Virus was released by freeze and thaw of cell cultures 24 h later and generation of virus progeny was determined by plaque assay.

B. Inhibition of miR-TS replication in EndoC-βH1 cells endogenously expressing miR-375. Human pancreatic EndoC-βH1 cells were infected at an MOI of 1 with H3N-39TS, H3N-375TS or H3N-375/1TS for 24 h, virus was released as described under A. and plaque assays were carried out.

FIG. 3: Replication and cytotoxicity of H3N-375TS and H3N-375/1TS in human colorectal carcinoma cell line DLD-1.

A. Growth kinetic. DLD-1 cells were infected at an MOI of 1 (upper diagram) or 0.01 (lower diagram) of the indicated viruses, samples were collected at 4 h, 24 h, 48 h and 72 h p.i. and the generation of virus progeny was measured by plaque assay.

B. Expression of CVB3 VP1 and cellular CVB3 target genes after infection. Left panel: DLD-1 cells were inoculated with indicated viruses at an MOI 10. Cells were analyzed after 24 h for CVB3 VP1 and cellular proteins eIF4G, cleaved eIF4G, caspase 3, cleaved caspase 3, PARP and cleaved PARP by Western blotting. The internal loading control was γ-tubulin. Mock: untreated cells. Right diagrams: Quantification of the expression of indicated genes was carried out relative to the expression of γ-tubulin by densitometric analysis using the ImageJ densitometry software (http://imagej.nih.gov/ij). aU, arbitrary units.

C. Cytotoxicity. Cells were infected with indicated viruses at an MOI of 1, 10 and 100 and cell viability was determined 24 h, 48 h and 72 h later with an XTT assay. Mock: untreated cells.

A. to C.: Data represent means±SEM of two independent experiments either in triplicate (A and C) or duplicate (B).

A., C.: Significance: * P<0.05; ** P<0.01 compared to cells treated with CVB3-H3. n.s., not significant.

FIG. 4: Biodistribution and replication of H3N-375TS and H3N-375/1TS in mice with subcutaneous DLD-1 cell tumors.

A. Replication of miR-TS viruses in different mouse tissues and DLD-1 cell tumors. DLD-1 cell tumors were established on both flanks of Balb/C nude mice (n=4 for each group) and intratumorally injected with 3×10⁶ pfu of indicated miR-TS virus when the tumor reached a diameter of ˜0.5 cm. Animals were sacrificed at day 4 after infection. The virus load was determined by plaque assay in the heart, spleen, liver, and brain and in the injected and not-injected contralateral tumors (left diagrams). In the pancreas, the virus copy number was determined by qRT-PCR (right diagrams). Note, at the time point of analysis all animals infected with H3N-39TS control virus were moribund, whereas there were no adverse effects seen in H3N-375TS- and H3N-375/1TS-infected mice. The contralateral not-injected tumor of one animal (each in H3N-375TS or H3N-375/1TS treated groups) did not develop. The data are shown for each animal and as medians for each group.

B. Histological examination of pancreas and heart. Tissue samples of the pancreas and the heart of sacrificed animals were fixed with formalin and stained with H&E. Images: Shown are representative slides of animals from each virus-infected group. Control: untreated animals. Arrows with open tops: Islets of Langerhans; Arrows with closed top: pancreas ducts; cross necrotic areas in the exocrine pancreas; black stars intact acinar cells of the exocrine pancreas. Diagram. The degree of pathological alterations in the pancreas and the heart was determined by a scoring system ranging from 0 (none) to 5 (high). The data are shown for each animal and as mean values for each group. With exception of H3N-39TS treated mice, which showed complete destruction of the pancreas, no pathological alterations were detected in the pancreas of virus-infected mice. Only in the heart of one H3N-375TS-infected mouse was tissue with marginal signs of tissue damage detected.

FIG. 5: Safety and oncolytic efficiency of H3N-375TS and H3N-375/1TS after long term treatment of mice with DLD-1 cell tumors. DLD-1 cell tumors were established in both flanks of Balb/C nude mice. When the tumor size reached ˜0.5 cm diameter, one of the tumors was injected with 3×10⁶ pfu H3N-375TS (n=4) or H3N-375/1TS (n=4). Two and four days after injection, virus injection was repeated using the same virus dose of 3×10⁶ pfu. Animals were sacrificed 35 days after first virus injection.

A. Tumor growth of H3N-375TS infected mice. Control: PBS-injected tumor; treated: virus injected tumor; untreated, contralateral uninjected tumor. The data are shown as mean values ±SEM for each group. Significance: ** P<0.01.

B. Data of A. shown for each animal.

C. Biodistribution and virus load H3N-375TS. Virus load was determined by plaque assay in the heart, spleen, liver, and brain and in the injected and not-injected contralateral tumors (left diagrams). In the pancreas the virus copy number was determined by qRT-PCR (right diagrams).

The data are shown for each animal and as medians for each group. Note, because of complete remission of three virus-infected tumors, the H3N-375TS titer was only determined in one tumor.

D. H3N-375TS titers in the blood of H3N-375TS infected mice. Note: M1 (mouse #1), M2 (mouse #2), M3 (mouse #3), M4 (mouse #4). Virus was not detected in the blood of M3 and M4.

E. Histological examination of pancreas and heart of H3N-375TS and H3N-375/1TS-infected mice. Tissue samples were prepared as in FIG. 4B. The images shown are representative of animals from the indicated groups. Control: untreated animals. There were no pathological alterations detected in either the pancreas or the heart of virus-infected animals.

F. Tumor growth of H3N-375/1TS-infected mice. Control: PBS-injected tumor; treated: virus injected tumor; untreated, contralateral un-injected tumor. The data are shown as mean values ±SEM for each group. Significance: * P<0.1, ** P<0.01.

G. Data of E shown for each animal.

H. Biodistribution and virus load H3N-375/1TS. Virus load was as determined and is presented as in C.

I. H3N-375/1TS titers in the blood of H3N-375/1TS infected mice. M1 (mouse #1), M2 (mouse #2), M3 (mouse #3), M4 (mouse #4). Virus was not detected in the blood of M4.

J. Kaplan-Meier survival curve. Significance: P=0.0041. Note: H3N-39TS-infected mice were moribund at day 4 after virus injection and were sacrificed according to the animal welfare guidelines.

K. Image of H3N-375TS-treated DLD-1 tumor mice. H3N-375TS treated mouse: arrow with closed top (left hand-side on the photo) shows non-injected tumor, arrow with open top (right hand-side on the photo)-shows site of virus injected tumor (note: promising there was no tumor detected in this mouse); Untreated control mouse: arrow with closed top (left hand-side on the photo) shows non-injected tumor, arrow with open top (right hand-side on the photo) shows tumor which was injected with PBS. Images were taken at day 29 after tumor cell injection.

FIG. 6: MiR-375 and miR-1 expression levels in harvested tumors and genetic stability of H3N-375TS and H3N-375/1TS.

A. Expression levels of miR-375 and miR-1 in H3N-375TS- and H3N-375/1TS injected DLD-1 tumors at day 35 after tumor inoculation.

The expression levels of miR-1 and miR-375 were determined by quantitative RT-PCR. Expression levels were normalized against U6 snRNA expression levels. Data are shown relative to the expression levels determined in in vitro cultured DLD-1 cells (set=1). Pancreas and heart tissues were harvested from PBS-treated control mice. Note: Investigated samples are from animals used in the experiment described under FIG. 4 and from animals used in the experiment described under FIG. 5.

B. Genetic analysis of miR-TS in H3N-375TS and H3N-375/1TS. Viral RNA was isolated from harvested tumor homogenates and the region containing the miR-TS was amplified by RT-PCR and cloned. MiR-TS of three clones was sequenced and compared with the sequences of H3N-375TS and H3N-375/1TS initially inserted miR-TS (termed here as consensus). The miR-TS are shown in bold+capital letters, and spacer sequences between the miR-TS in italics and small letters. Nucleotide substitutions are underlined. Stuffer sequences upstream of the 5′ miR-TS copy are shown in italics and small letters (indicated by “Stuffer”). The first three nucleotide at the 5′ end of the sequence represent the stop codon of open reading frame of the viral polyprotein encoding sequence. Note: Investigated samples are from animals used in the experiment described in FIG. 5. Upper panel (nucleotide sequence): Consensus miR-375TS construct—SEQ ID No: 16; H3N-375TS, 32 days pi (post implantation of) tumor: 1.—SEQ ID No: 17; 2.—SEQ ID No: 18; 3.—SEQ ID No: 19. Lower panel (nucleotide sequence): Consensus miR-375TS/miR-1TS construct—SEQ ID No: 20; H3N-375TS/1TS, 32 days pi (post implantation of) tumor: 1.—SEQ ID No: 21; 2.—SEQ ID No: 22; 3.—SEQ ID No: 23.

FIG. 7: Tissue distribution of H3N-375TS and H3N-375/1TS.

DLD1 cell tumors were established in both flanks of Balb/c nude mice. When tumor size reached ˜0.5 cm diameter, one of the tumors was injected with single dose of 3×10⁶ pfu H3N-375TS (n=6) or H3N-375/1TS (n=6). Animals were sacrificed 10 days (H3N-375TS) and 20 days (H3N-375/1TS) after virus injection.

A. Virus biodistribution. Virus load was determined by plaque assay in the heart, spleen, liver, and brain and in the injected and the contralateral non-injected tumors. In the pancreas the virus copy number was determined by qRT-PCR. The data are shown for each animal and as medians for each group.

B. Histological examination of pancreas and heart. Images: Tissue samples of the pancreas and the heart of sacrificed animals were fixed with formalin and stained with H&E. Shown are representative slides of animals from both virus-infected groups. Note: The upper images from heart and pancreas show intact tissue without pathological alterations. The lower image is from an animal which has infiltration of inflammatory cells (arrows) in the heart. Diagram: The degree of pathological alterations in the pancreas and the heart was determined by a scoring system ranging from 0 (none) to 5 (high). Data are shown for each animal and as mean values for each group.

### EXAMPLES

The following examples illustrate viable ways of carrying out the invention as intended, without the intent of limiting the invention to said examples.

### Cell Lines

HeLa cells were cultured in Dulbecco's modified Eagle's medium (DMEM) (Gibco, Karlsruhe, Germany) supplemented with 5% fetal calf serum (FCS) and 1% penicillin-streptomycin. HEK293T cell line was cultured in DMEM High Glucose (Biowest, Darmstadt, Germany) supplemented with 10% FCS, 1% penicillin-streptomycin, 1% L-glutamine and 1 mM Na-pyruvate. Colorectal carcinoma cell line DLD-1 was grown in RPMI 1640 supplemented with 10% FCS, 1% penicillin-streptomycin, 1% L-glutamine and 1 mM Na-pyruvate (Invitrogen, Karlsruhe, Germany). Human insulinoma Endoc-βH1 cells were cultured as described previously (Scharfmann et al. 2014). Embryonic mouse cardiomyocytes (EMCM) were obtained from C57BL/6 mice on embryonic day 14 and cultured as described previously (Spur et al. 2016).

### Viruses

CVB3 strain H3 was generated by transfection of the cDNA containing plasmid pBK-CMV-H3 (kindly supplied by Andreas Henke, Institute of Virology and Antiviral Therapy, University of Jena, Jena, Germany) into HEK293T cells using Polyethylenimine Max (Polysciences, Inc., Warrington, PA). Generation and production of H3N-375TS and H3N-39TS have been previously described (Pinkert et al. 2020); cf. methods, 2.1 and FIG. 2 and miR-375TS construct 3′UTR and 5′UTR cf. additional (Pryshliak et al. 2020), Materials and methods, plasmid and viruses part). H3N-375/1TS, which encodes two copies each of miR-375TS (5′-TCACGCGAGCCGAACGAACAAA-3′, SEQ ID No: 1) and miR-1TS (5′-ATACATACTTCTTTACATTCCA-3′, SEQ ID No: 2), was constructed by insertion of two copies of miR-1TS into the 3′UTR of the H3N-375TS genome in place of last copy of the miR-375TS. The miR-1TS sense primer (5′-TCCAAGGCCTATATACATACTTCTTTACATTCCATTAGAGACAATTTGATCTGATTTGA-3′, SEQ ID No: 24; underline indicates miR-1TS sense) and antisense primer (5′-TATATAGGCCTTGGAATGTAAAGAAGTATGTATGCGCTTTGTTCGTTCGGCT-3′, SEQ ID No: 25; underline indicates miR-1TS antisense) were designed using the online Infusion primer designing tool (Takara Bio, Japan) and cloning was done by In-Fusion HD Cloning Kit (Takara Bio) according to manufacturer's instructions using the plasmid pMKS1-H3N-375TS ((Pinkert et al. 2020); cf. methods, 2.1 and FIG. 2) which contains the cDNA of H3N-375TS. The resulting plasmid was termed pMKS1-H3N-375/1TS. In vitro T7 transcription kit (Roboklon GmbH, Berlin, Germany) was used to obtain viral RNA from pMKS1-H3N-375TS and pMKS1-H3N-375/1TS. Two μg of the viral RNA was transfected into HEK293T cells and once complete cell lysis was observed, cell plates were stored in −80° C. Following three freeze and thaw cycles, cell debris was cleared by centrifugation. To obtain higher a titer, all viruses were amplified in HeLa cells. For in vivo experiments, viruses were purified and concentrated in sucrose gradient as previously described (Pinkert et al. 2020).

### Virus Plaque Assays

Virus plaque assays were carried out as described previously (Fechner et al. 2008). Briefly, HeLa cells were cultured in 24-well culture plates as confluent monolayers. After 24 h, medium was removed and cells were overlaid with serial ten-fold (−2 to −8) diluted supernatant harvested from homogenized mouse organs, followed by 3 freeze/thaw cycles and then incubated at 37° C. for 30 min and, after removal of the supernatant, overlaid with agar containing Eagle's minimal essential medium (MEM). Three days later, the cells were stained with 1×3-(4,5-dimethylthiazol-2-yl)-2,5-diphenyl tetrazolium bromide iodotetrazolium chloride (MTT/INT) solution. Virus titers were determined by plaque counting one hour after staining.

### Growth Curves

HeLa (1×10⁶) and DLD-1 (1×10⁶) were seeded into 6 well plates for full confluency and after 24 h, cells were infected for 1 hour at an MOI (multiplicity of infection) of 0.1 (HeLa cells) or an MOI of 1 or 0.01 (DLD-1 cells), respectively. Afterwards, virus solutions were removed, and cells were washed with PBS. Two ml fresh medium was added, and cell plates were incubated at 37° C. and 5% C02. Plaque assays were performed for virus titration by collecting 100 μl supernatant 4 h, 24 h, 48 h and 72 h post-infection) p.i..

### MiR Expression Analysis

Total RNA from cells or mouse tissues were isolated using Life Technologies TRIZOL reagent according to the manufacturer's instructions. Total RNA was digested with DNAse I (Peqlab, Erlangen, Germany) and reverse transcribed using the High-Capacity cDNA Reverse Transcription Kit (Applied Biosystems, Foster City, CA, USA). Expression levels of miR-375 (assay ID; 000564), miR-1 (assay ID; 002222), miR-34 (assay ID; 000426) and miR-16 (assay ID; 000391) were determined by utilizing the TaqMan gene expression master mix and specific TaqMan gene expression assays from Life Technologies according to manufacturer's instructions. Real-time PCR was performed using a CFX96 Real-Time System combined with a C1000 Thermal Cycler (Bio-Rad). The data was analyzed by using ΔΔCT method and results were normalized against U6 snRNA (assay ID; 001973) levels of cell lines and tissues.

### Genetic Stability of MiR-TS

Viral RNA was isolated with High Pure viral nucleic acid kit (Roche, Mannheim, Germany) from harvested tumor or tissue homogenates according to the manufacturer's protocol. Following DNase I digestion (Peqlab, Erlangen, Germany), viral RNA was reverse transcribed using a high-capacity cDNA reverse transcription kit (Applied Biosystems Inc., Foster City, CA) with antisense primer (5′-CTACTGCACCGTTGTCTAG-3′, SEQ ID No: 26). Afterwards PCR was performed with sense (5′-CCATAGATGCGTCTTTGCT-3′, SEQ ID No: 27) and antisense primers (5′-CCGTTGTC TAGTTCGGTT-3′, SEQ ID No: 28) to amplify the region from nucleotides 6923 to 7374 of the viral genome which contains miR-TS. The PCR fragments were subcloned into a plasmid using CloneJET PCR Cloning Kit (Thermo Fisher Scientific) according to manufacturer's protocol. Sequencing made use of the primer: 5′-CAGGAGCGTCCCAGTTGG-3′ (SEQ ID No: 29).

### Western Blots

Western blots were carried out as previously described (Pryshliak et al. 2020). Briefly, cells were lysed with buffer containing 20 mM TRIS/HCl, pH 8.0, 140 mM NaCl, 1 mM EDTA, 1% Triton X-100, 1% protease inhibitor cocktail (Sigma-Aldrich, Taufkirchen, Germany) and 1% phosphatase inhibitor cocktail (Calbiochem, San Diego, CA, USA), the protein concentration was measured with a BCA assay (Thermo Fisher Scientific), and cell extracts were separated by SDS-PAGE. Immunoblots were carried out with primary anti-gamma-tubulin antibody from Sigma-Aldrich and anti-eIF4G, cleaved caspase 3 and anti-PARB antibodies from Cell Signaling Technology (Danvers, MA, USA). The monoclonal anti-VP1 antibody was generated against VP1 from CVB5 strain Faulkner. Relative Quantification of gene expression was carried out by densitometric analysis using the ImageJ densitometry software (http://imagej.nih.gov/ij).

Virus Silencing in HEK293T Cells Transfected with miRs

Sixty percent confluent HEK293T cells were transfected with 800 ng miR expression plasmids; pCMV-miR-1 expressing the miR-1, pCMV-miR-375 expressing the miR-375 and pCMV-miR expressing only GFP as control (Origene Technologies, Rockville, MD, USA) with PEI Max transfection reagent. GFP signal was monitored with fluorescent microscope for transfection efficiency. The medium was discarded 24 h after transfection and cells were inoculated with viruses (MOI of 0.1) for 30 minutes at 37° C. Following removal of viral solutions, fresh medium was added. Cells were subjected to 3 freeze/thaw cycles 24 h post virus infection and the cell lysate was centrifuged to remove cell debris. The supernatant was used for determination of virus titers by plaque assay.

### Cell Viability

Cell viability was assessed using Cell Proliferation Kit (XTT) (Promega GmbH, Walldorf, Germany) according to manufacturer's instructions. Briefly, cells were seeded onto 96-well plate and were 25 infected at an MOI of 1, 10 or 100. At the indicated time points, absorbance levels were measured using a V-650 Spectrophotometer (Jason Inc. Milwaukee, WI, USA). As a negative control, cells were treated with 5% Triton X-100 solution.

### Histopathological Analysis

The mouse tissues and explanted human tumors were fixed in 4% paraformaldehyde, embedded in paraffin. Five μm thick tissue sections were cut and stained with hematoxylin and eosin (H&E) to visualize and quantify cell destruction and inflammation. Damage of the pancreas and the heart was determined by a scoring system (0—no detectable pathological changes to 5—extensive pathological changes in the entire tissue) which includes infiltration with immune cells, necrosis, lesion area, cellular vacuolization and calcification in the organs as described previously (Wang et al. 2019).

### In Vivo Experiments

Animal experiments were performed in accordance with the principles of laboratory animal care and all German laws regarding animal protection. Human colorectal DLD-1 cells (5×10⁶ cells) were xenografted subcutaneously into the right and left flanks of 6-week old female BALB/c nude mice. Tumor burdens were measured daily by hand caliper. One of the tumors was intratumorally injected with 3×10⁶ pfu of virus when tumor size reached 0.4-0.5 cm in diameter. For short term investigations, animals received a single dose of virus and were sacrificed 4 days, 10 days or 20 days p.i.. For long term study, animals were injected three times on days 0, 2 and 4 and investigated 32 days after the first virus injection. The control mice were intratumorally injected with PBS.

### Statistical Analysis

Statistical analysis was performed with Graph-Pad Prism 8.2 (GraphPad Software, Inc., La Jolla, CA, USA). Results are expressed as the mean±SEM for each group. Statistical significance was determined by use of the two-tailed unpaired Student t-test for cell culture investigations and by use of the Mann-Whitney U-test for in vivo investigations. Survival curves were plotted according to the Kaplan-Meier method and statistical significance determined by the (log-rank-test). Differences were considered significant at p<0.05.

### Experimental Description

Determination of an appropriate miR is a crucial step to achieve adequate miR-detargeting. Most importantly, the miR should be weakly expressed or absent in the tumor but abundantly expressed in the healthy organs where undesirable virus replication takes place. Recently it has been shown that miR-34a, a tumor suppressor miR fulfills this requirement and an engineered CVB3 with corresponding miR-34aTS was successfullydetargeted from the pancreas and the heart in a murine model of lung cancer (Jia et al., 2019). However, heterogeneity of cancer may cause variable expression of tumor suppressor miRs. The inventors of the present invention have found high expression of miR-34a in colorectal cancer. Moreover, the expression levels of miR-34a were similar to those in the pancreas and the heart (FIG. 1F) making most probably a miR-34a detargeting strategy unsuitable for colorectal cancer as it would be detargeted not only in the pancreas and the heart but also in the colorectal cancer cells. Therefore, the inventors of the present invention set themselves the task to find another approach to detarget CVB3 from the pancreas and the heart. In former studies of the inventors they could show that Colorectal cancer cell culture lines infected with a CVB3, which was equipped with miR-TS complementary to the miR-375 in vitro showed high replication and induced tumor cell cytotoxicity, wherein the grade was more pronounced when the miR-TS was inserted at the 3′UTR of the polyprotein coding region of the CVB3 transcript when compared to its the 5′UTR MiR-375 was found the most abundantly expressed miR in the human pancreas tissue, but weakly expressed in colorectal carcinoma cell lines. In (Pinkert et al. 2020) the inventors were able to show that when a CVB3, which was equipped with miR-TS complementary to the miR-375, was intraperitoneal administered to NMRI mice (without any tumor transplant association) that these mice did not result in pancreatic infection. However, depending on the mode of treatment the mice could develop cardiac CVB3 infection. This was the case when the CVB3 was administered intravenously: The respective animals developed moderate myocarditis resulting from virus infection of the heart. Cardiac virus titers reached 2.6×10⁴ pfu/g. Moreover, intravenous application of H3N-375TS to NMRI mice also led to chronic myocarditis, which is characterized by myocardial fibrosis and persistence of the CVB3 RNA genomes bearing the miR-375TS but very promising absence of replicating virus. When the virus was administered intraperitoneally a myocarditis could even more promisingly not be detected. Without being bound by this hypothesis the reason for this is believed to be the following: The pancreas is the most susceptible organ for CVB3 in mice and primary site of CVB3 infection from which the virus spread to other organs. Thus, these former observations indicate that blocking the virus replication in the pancreas (when the mode of CVB application is intraperitoneal) is the major cause for preventing infection of other organs as the heart and to increase safety of therapeutic CVB3s. Accordingly, there is a major interest to further increase the safety of therapeutic CVB3 by introducing additional modifications to restrict the replication specific to tumor cells to be targeted. And this strategy should be ideally independent of the mode of administering to prevent particular besides the pancreas especially also the cardiac virus infection and myocarditis.

### Example 1: Pancreas-Specific miR-375 and Cardiac-Specific miR-1 are Downregulated in Colorectal Cancer Cell Lines

For selective silencing of miR-TS equipped oncolytic viruses, the corresponding miR should be highly expressed in the tissues where viral replication must be suppressed, whereas its expression should be low or absent in the targeted tumor/cancer cells. Here we focused on the pancreas specifically expressed miR-375. To further strengthen safety measures and guarantee that the infectious construct will not infect heart tissue the heart-specifically expressed miR-1 expression was also tested. First, to compare pancreatic and cardiac expression of both miRs with expression levels in colorectal carcinomas, seven colorectal carcinoma cell lines, murine pancreas and heart tissues, as well as the pancreatic cell line EndoC-βH1 and embryonic mouse cardiomyocytes (EMCM), were investigated for expression of miR-375 and miR-1. Using quantitative RT-PCR, we found that miR-375 was highly expressed in the pancreas and EndoC-βH1 cells, and weakly expressed in colorectal carcinoma cell lines, murine heart and EMCM (at least 200-fold lower compared to the pancreas). MiR-1 was strongly expressed in the heart and expressed about 40-fold weaker in EMCM compared to the heart, and at least 400-fold more weakly expressed in the pancreas and in the colorectal carcinoma cell lines. We also found that both miRs were weakly expressed in murine spleen, liver and brain, which is important, as CVB3 can also infect these tissues. Moreover, HeLa and HEK293T cells which are used for virus production expressed miR-375 and miR-1 at very low levels (FIG. 1A). Hence, both miRs fulfilled the essential requirements for use as silencers of a bioengineered oncolytic CVB3. High expression of miR-375 and miR-1 in mouse pancreas and heart, respectively, as well as low expression or absence of these miRs in colorectal carcinoma cells was confirmed in this study. Thus, both miRs fulfilled the most essential requirements to be used as inhibitors of CVB3 replication.

### Example 2: Insertion of miR-TS into the CVB3 Genome does not Affect Viral Replication in HeLa Cells

Without being bound by this theory previous observations in mice suggest that the pancreas is the primary site of CVB3 replication essential for the distribution of the virus via the blood stream and subsequent cardiac CVB3 infection. Accordingly, we hypothesized that the pancreas, and the heart, may be protected from CVB3 infection, when the viral replication in the pancreas is suppressed by pancreas-specific miRs. To prove this, we used H3N-375TS as described in (Pinkert et al. 2020), a variant of the CVB3 strain H3 containing three copies of the miR-375TS recently engineered by our group. In addition, we newly developed H3N-375/1TS, which contains two copies of miR-375TS and two copies of miR-1TS in the H3 backbone. With the additional insertion of miR-1TS, we expected that viral replication in the heart in certain circumstances would be strongly inhibited than replication of H3N-375TS. In both viruses the miR-TS was inserted into the 3′UTR of virus genome, immediately downstream of the stop codon of the CVB3 polyprotein encoding sequence (FIG. 1B), as we and others have found that this region tolerates miR-TS well. Both, miR-375TS and miR-1TS were 100% complementary to their corresponding miR-375 and miR-1 with respect to hypothetical nucleotide-basepairing (cf. FIG. 1C), respectively.

To assess whether miR-TS insertion affects growth perse of the engineered viruses we determined their growth kinetics in highly susceptible HeLa cells over 72 h and compared them with growth kinetics of CVB3-H3 and the control virus H3N-39TS bearing miR-TS of the cel-miR-39, which is not expressed in mammalian cells. As shown in FIG. 1D, there are beneficially no differences in viral growth. All viruses grew rapidly and reached a plateau already by 24 h after infection. Moreover, virus plaques sizes were similar for all viruses (FIG. 1E). This positively indicates that viral replication was unaffected in this cell line despite the insertion of miR-TS.

### Example 3: H3N-375TS and H3N-375/1TS are Susceptible for Cognate miRs

To investigate whether replication of H3N-375TS and H3N-375/1TS can be inhibited by cognate miRs, we first transfected HEK293T cells with miR-375-, miR-1- or an GFP-expressing control plasmid and infected the cells 24 h later with 0.01 MOI of H3N-375TS, H3N-375/1TS or the control virus H3N-39TS for 24 h. H3N-375TS was inhibited by 8.4-fold in cells transfected with miR-375, but remained unaffected in miR-1-transfected cells, whereas H3N-375/1TS was inhibited in both miR-375- and miR-1-transfected cells by 17.7-fold and 11.3-fold, respectively. H3N-39TS replication was neither suppressed in miR-375-nor in miR-1-transfected cells (FIG. 2A).

Having demonstrated that transiently expressed miR-375 and miR-1 specifically inhibit H3N-375TS and H3N-375/1TS, respectively, we next investigated whether replication of the viruses is also suppressed in cells expressing the miR-375 and miR-1 endogenously (FIG. 1A). Therefore, EndoC-βH1 cells were infected at an MOI of 1 and EMCM with a MOI of 0.01 of H3N-375TS, H3N-375/1TS or H3N-39TS. The viral titers were measured 24 h later by plaque assay. In EndoC-βH1 cells, the H3N-39TS propagated robustly, resulting in generation of virus titers of ˜10⁷ pfu/ml, whereas replication of H3N-375TS and H3N-375/1TS was significantly lower, reaching only ˜10¹ pfu/ml (FIG. 2B). In EMCM, H3N-375TS titers were unchanged compared to H3N-39TS (˜4.3×10⁴ pfu/ml), whereas the titers of H3N-375/1TS were almost two orders of magnitude lower (˜6×10² pfu/ml) (FIG. 2C). The distinctly higher inhibition of H3N-375TS and H3N-375/1TS in EndoC-βH1 cells compared to miR-375 transfected HEK293T cells can be explained by the fact that in the latter only 60% of cells were transfected with the miR-375 expression plasmids (determined by a GFP reporter), respectively, whereas in EndoC-βH1 all cells endogenously express the miR-375. Therefore, only some of the HEK293T cells were protected against the viruses, whereas in the untransfected cells the viruses freely replicated. This leads to higher virus titers in miR-375 transfected HEK293T cells and less inhibition of virus replication compared to EndoC-H1 cells. However, as indicated by comparably low inhibition of H3N-375/1TS in EMCM, which expresses the miR-1 endogenously, cell type specific differences may also play a role in explaining differences in the strength of miR-induced virus inhibition.

Taken together, these results clearly demonstrate that H3N-375TS and H3N-375/1TS are efficiently and specifically suppressed in cells expressing cognate miR-375 and miR-1.

### Example 4: Insertion of miR-TS Slightly Reduces Growth and Cytotoxicity of H3N-375TS and H3N-375/1TS in the Colorectal Carcinoma Cell Line DLD-1

The human colorectal carcinoma cell line DLD-1 is highly susceptible to CVB3-H3 18 and expresses miR-375 and miR-1 at low levels (FIG. 1A) which makes this cell line suitable to demonstrate the oncolytic potential of H3N-375TS and H3N-375/1TS. First of all, we determined the growth kinetics of both viruses and compared them with those of H3N-39TS and CVB3-H3 control viruses in these cells. As shown in FIG. 3A (upper diagram), at a high MOI of 1 all viruses grow rapidly, reaching a plateau after 48 to 72 h and showed similar growth curves. However, when the virus dose was reduced to an MOI of 0.01 (FIG. 3A, lower diagram) differences in virus replication kinetics became apparent. In fact, the three H3N-TS viruses showed a lower proliferation rate than CVB3-H3. Replication activity of H3N-375TS and H3N-375/1TS in DLD-1 cells was confirmed by investigation of CVB3 VP1 and CVB3 target-gene expression by Western blotting. As shown in FIG. 3B, VP1, cleaved eIFG4, caspase 3 and PARP were upregulated in H3N-375TS, H3N-375/1TS, H3N-39TS and CVB3-H3 infected cells, but there were no significant differences between the viruses with respect to these proteins (FIG. 3B).

Cytotoxic activity represents a second important feature of oncolytic viruses. We therefore next investigated H3N-375TS- and H3N-375/1TS-induced cell killing activity in DLD-1 cells. DLD-1 cells were infected with 1 to 100 MOI of either viruses or with H3N-39TS and CVB3-H3 and cell viability was determined by XTT assay over a 72 h period. There were no differences in virally induced cytotoxicity at 24 h and 48 h after infection at each applied dose, as well as at 72 h after infection with a virus dose of 1 MOI. However, a significantly lower cytotoxicity of all miR-TS viruses compared to CVB3-H3 became apparent at 72 h when the cells were infected at an MOI of 10. Cell viability reached only 11% for parental CVB3-H3, whereas it reached 42% for H3N-375TS, 39% for H3N-375/1TS and 29% for H3N-39TS (FIG. 3C).

Taken together, the results show that insertion of miR-375TS and miR-1TS into the genome of a CVB3 virus as CVB3-H3 beneficially only slightly impairs viral replication, interaction of the virus with cellular targets and the virus-induced cytotoxicity in DLD-1 colorectal carcinoma cells.

Additionally, our in vitro investigations of H3N-375TS and H3N-375/1TS confirmed susceptibility of both viruses to their miR-TS cognate miRs. However, both viruses showed a slight lower replication and cytotoxicity in colorectal DLD-1 cancer cells compared to the parental CVB3-H3 strain. As reduced replication and cytotoxicity was also seen with miR-TS control virus, we assume that the insertion of miR-TS into the viral genome is itself responsible for this, rather than a result of specific silencing effect induced by miR-1 and/or miR-375, which are expressed at very low levels in this cell line.

### Example 5: In Vivo H3N-375TS and H3N-375/1TS are Detargeted from Mouse Tissues Expressing Cognate miRs

To investigate safety and oncolytic activity of H3N-375TS and H3N-375/1TS in vivo, we established subcutaneous DLD-1 cell tumors in both flanks of in nude mice and injected one tumor with 3×10⁶ pfu H3N-375TS, H3N-375/1TS or control virus H3N-39TS, when the tumors reached a size of ˜0.5 cm. H3N-39TS-infected mice were sacrificed four days after virus injection, when the animals became moribund. As expected, the mice had high amounts of virus in the heart and the pancreas and in the injected and contralateral tumor. Moreover, moderate H3N-39TS levels were found in the spleen, liver and brain (FIG. 4A). Histological examination confirmed complete damage of the pancreas, whereas pathological alterations were not detectable in the heart (FIG. 4B) or in other organs (results not shown). In H3N-375TS-infected animals, only the injected and the contralateral tumor showed high virus titers, which, however, were promising ˜10 to 30-fold lower than in H3N-39TS-infected mice. Importantly, the pancreas of the animals was virus free, and virus titers in the heart were much lower (˜2,000-fold) than in the heart of H3N-39TS-infected mice. Furthermore, of note the spleen and liver were also virus free in the H3N-375TS approach, and only one of four animals showed detectable virus in the brain (FIG. 4A). Virus distribution and titers in H3N-375/1TS-infected mice were similar to those in H3N-375TS-infected mice, except that very advantageously next to the pancreas also the heart was virus free (FIG. 4A). Accordingly, with exception of one H3N-375TS-infected mouse, which showed very weak cardiac inflammation (results not shown), the heart and the pancreas of H3N-375TS- and H3N-375/1TS-infected mice as well the other organs of these mice (results not shown) did not show any pathological alterations under the histological examination (FIG. 4B).

Accordingly, these data demonstrate that H3N-375TS was specifically suppressed in the pancreas and H3N-375/1TS in the pancreas and in the heart of tumor infected mice. Moreover, tissue-specific miR-375- and miR-1-mediated inhibition of H3N-375TS and H3N-375/1TS replication also very promisingly reduced virus burden in tissues which even did not express miR-375 or miR-1 (cf. FIG. 4A).

### Example 6: In Vivo H3N-375TS and H3N-375/1TS Efficiently Inhibit Growth of Colorectal Carcinomas in Mice, without Inducing Side Effects

Having shown inhibition of H3N-375TS and H3N-375/1TS replication in the pancreas and heart of DLD-1 tumor bearing mice shortly after infection, we next investigated oncolytic activity and safety of both viruses in a long-term therapeutic approach. Tumor bearing mice received intratumoral virus administration (3×10⁶ pfu per dose) when tumors reached a size of ˜0.5 cm and again two and four days after the first injection. The animals were sacrificed at day 32 post-initial-injection. Treatment with H3N-375TS led to complete regression of the injected tumor in three of the four mice and to partial regression in the remaining animal (FIG. 5A, K). A significant inhibition of growth of the non-injected contralateral tumors was also observed when compared to tumors in untreated control animals, but inhibition of tumor growth was less pronounced than of the infected tumor (FIG. 5B, K). Analysis of virus burden showed low H3N-375TS titers in three of the four non-injected contralateral tumors, whereas the only one growing injected tumor and the normal organs were virus free (FIG. 5C). Two out of four animals had viremia. Viremia is a medical condition where viruses enter the bloodstream and hence have access to the rest of the body. However, the serum titers of said two animals were low (FIG. 5D). Interestingly and of note: No virus-related adverse effects were observed in the animals during the period of observation and histological examination excluded heart and pancreas damage and inflammation, respectively (FIG. 5E).

H3N-375/1TS-infected mice also showed significant inhibition of growth of the injected and contralateral non-injected tumors. However, growth inhibition was slightly weaker than in H3N-375TS-infected mice and there was no complete tumor regression (FIG. 5F, G). All of the injected tumors and also two of four non-injected contralateral tumors had low virus titer (FIG. 5H). Viremia was detected in three of four animals (FIG. 5I), but the titers were slightly higher than in H3N-375TS infected mice. As observed in H3N-375TS-infected mice, H3N-375/1TS-injected mice also did not show virus-related adverse effects and the pancreas and the heart were free of pathological alterations (FIG. 5E). Importantly and promising, there was no mortality in mice infected H3N-375TS and H3N-375/1TS, so that the overall survival time was significantly prolonged compared to mice receiving only the control virus H3N-39TS (FIG. 5J). We also investigated H3N-375TS and H3N-375/1TS-infected animals, which were analyzed 10 and 20 days post infection (p.i.), respectively. In agreement with the data obtained at 32 days after infection, H3N-375TS-infected mice had no virus in the pancreas and low titers in the heart, whereas in H3N-375/1TS-infected mice virus titers in the pancreas and the heart were undetectable (FIG. 7).

In vivo all treated animals beneficially survived until the scheduled end of the experiment at day 32 post-intratumoral virus injection, and no virus-induced sickness was detected in this period at all. As the miR-TS control virus killed animals within four days, the dramatic difference in animal survival is apparent. Safety of H3N-375TS and H3N-375/1TS was obviously caused by preventing viral replication in the pancreas and the heart. In fact, H3N-375/1TS was not detected in both organs, confirming that pancreatic and cardiac replication of H3N-375/1TS were successfully inhibited by the miR-375 and miR-1. H3N-375TS was also ablated from the pancreas, but interestingly the H3N-375TS titer was also reduced in the heart, even though the virus is not susceptible to miR-1. Similarly, the spleen, liver and brain of H3N-375TS- and H3N-375/1TS-infected animals were (with few exception) virus free, whereas high titers where detected in animals which were infected with the miR-TS control virus. Based on low miR-375 and miR-1 expression levels in these organs, we exclude miR-induced inhibition as the cause for the inhibition. Both viruses showed significant oncolytic activity in vivo. However, whereas three out of four H3N-375TS-injected DLD-1 cell tumors showed complete regression, tumor clearance was not seen in H3N-375/1TS-injected animals, indicating a lower oncolytic activity of the latter.

### Example 7: MiR-1 Expression is Strongly Increased in DLD-1 Tumor Bulk Compared to DLD-1 Monolayers

There was no difference between H3N-375TS and H3N-375/1TS in growth kinetics and cytotoxicity in DLD-1 cells in vitro, which rules out that the intrinsic activity of H3N-375/1TS is lower than that of H3N-375TS. As shown above DLD-1 tumor destruction was lower in H3N-375/1TS than in H3N-375TS infected mice. To elucidate whether this could be related to increase of miR-1 levels in the growing tumors, we determined miR-1 expression in DLD-1 tumor bulk harvested at day 32 after the first virus injection and compared it with miR-1 expression in a DLD-1 cell monolayer and in the heart. As shown in FIG. 6A, distinctly more miR-1 was indeed expressed in the DLD-1 tumor bulk than DLD-1 monolayers, ranging from 125-fold in untreated tumors to 675-fold and 540-fold in H3N-375TS and H3N-375/1TS injected tumors, respectively. However, even the highest miR-1 levels were still greater than 3 orders of magnitude below the levels measured in the heart. We also investigated miR-375 expression in DLD-1 tumors and DLD-1 monolayers. Only a slight increase of 3- to 5-fold was detected in the tumor and the absolute miR-375 level remained greater than 100-fold below the levels in the murine pancreas (FIG. 6A). Thus, considering other potential inhibitory factors, we found that miR-1 was strongly induced in DLD-1 cell tumors by 125-fold compared to DLD-1 cell culture. Moreover, in virus-infected DLD-1 tumor masses, the miR-1 levels were elevated more than 500-fold.

These data demonstrate that miR-1 was strongly upregulated in the established tumors, but compared to its expression in the heart the expression remained low. Thus, a selective inhibition of H3N-375/1TS by endogenously upregulated miR-1 in DLD-1 cell tumors seems to be the most plausible explanation for lower oncolytic efficacy of H3N-375/1TS compared to H3N-375TS in vivo.

### Example 8: H3N-375TS and H3N-375/1TS Show High Genetic Stability of Both miR-TS Sequences in the Equipped CVB3 cDNA Construct

Lack of adverse effects and lack of replication of H3N-375TS in the pancreas and of H3N-375/1TS in the pancreas and the heart suggests high stability of both miR-TS viruses. To prove this, we cloned and sequenced the miR-TS box of each three clones of H3N-375TS and H3N-375/1TS which were isolated from the injected tumors at day 32 after the first virus injection. In one H3N-375TS clone the miR-375TS box was completely intact, whereas in the other two clones four and two nucleotides were mutated each in one of the three miR-TS copies, respectively. Most promisingly, in H3N-375/1TS, only one nucleotide substitution in one miR-1TS of one clone was detected, whereas the other miR-375TS and miR-1TS copies were intact (FIG. 6B), indicating a high genetic stability.

Surprisingly, in the context of the present invention these data indicate that H3N-375TS and H3N-375/1TS show high genetic stability of both respective miR-TS sequences in the equipped CVB3 cDNA construct, the H3N-375/1TS even a more pronounced one.

### Example 9: In Vitro and In Vivo Data

To investigate the importance of the microRNAs, two viruses were engineered as described above, H3N-375TS containing only miR-375TS and H3N-375/1TS containing miR-375TS and miR-1TS. In vitro, both viruses replicated in and lysed colorectal carcinoma cells, similar to a non-targeted control virus H3N-39TS, whereas they were strongly attenuated in cell lines transiently or endogenously expressing the corresponding microRNAs.

In vivo, the control virus H3N-39TS induced strong infection of the pancreas and the heart which led to fatal disease within four days after a single intratumoral virus injection in mice xenografted with colorectal DLD-1 cell tumors. In contrast, three intratumoral injections of H3N-375TS or H3N-375/1TS failed to induce virus-induced sickness. In the animals, both viruses were completely ablated from the pancreas and H3N-375/1TS was also ablated from the heart, whereas the cardiac titers of H3N-375TS were strongly reduced. Long term investigations of the DLD-1 tumor model confirmed lack of virus-induced adverse effects in H3N-375TS and H3N-375/1TS treated mice. There was no mortality and the pancreas and the heart were free of pathological alterations. Regarding the therapeutic efficiency, the treated animals showed high and long-lasting H3N-375TS and H3N-375/1TS persistence in the tumor and significantly slower tumor growth. In overall conclusion these data confirm the pronounced safety of H3N-375/1TS and H3N-375TS in vivo.

Importantly, both equipped viruses showed high oncolytic activity, which however was slightly higher for H3N-375TS than for H3N-375/1TS. These data give clear indication for improved safety characteristics of CVB3 equipped with miR-375TS and miR-1TS for application in the anti-tumor therapy in humans. Moreover, these data advantageously demonstrate that tissue-detargeting by use of pancreas- and heart specific miR-TS as miR-375 and miR-1, respectively, is a highly effective strategy to prevent off-site toxicity of oncolytic CVB3 and to increase tumor selectivity of oncolytic CVB3, which may be suitable for use in other oncolytic CVB3 strains.

1. An infectious complementary DNA (cDNA) construct characterized in that it comprises:
the genomic sequence of a Coxsackievirus B3 (CVB3);
at least one or more microRNA target sequences (miR-TS), which are complementary to one or more microRNAs having tissue-specific expression pattern,
wherein the at least one or more miR-TS are integrated adjacent of the 5′UTR and/or the 3′UTR of the CVB3 protein coding sequence.

2. Infectious cDNA construct according to claim 1, characterized in that the cDNA construct is in the form of a plasmid.

3. Infectious cDNA construct according to claim 1 or 2, characterized in that the at least one or more miR-TS are incorporated between the stop codon of the coding sequence of the 3D polymerase and the 3′UTR of the CVB3 protein encoding sequence, optionally wherein the at least one or more miR-TS are flanked by a stuffer sequence.

4. Infectious cDNA construct according to any of the previous claims, characterized in that the at least one or more miR-TS are complementary to miR sequences, which are specifically expressed in the human pancreas tissue and/or are complementary to miR sequences, which are specifically expressed in the human heart tissue.

5. Infectious cDNA construct according to any of the previous claims, characterized in that the at least one or more miR-TS comprise or consist of a first miR-TS, which is complementary to a miR sequence, which is specifically expressed in the human pancreas tissue and a second miR sequence, which is specifically expressed in the human heart tissue.

6. Infectious cDNA construct according to any of the previous claims, characterized in that the at least one or more miR-TS are complementary to a miR sequence selected from the group consisting of human pancreas tissue specific expressed miRs: miR-375, miR-690, miR-375, miR-217, miR-216a, miR-216b, miR-200a, miR-200b, miR-200c, miR-429, miR-141 and/or human heart tissue specific expressed miRs: miR-1, mriR-133, miR-206.

7. Infectious cDNA construct according to claim 5, characterized in that
the at least one or more miR-TS are complementary to a miR sequence selected from the group consisting of human pancreas tissue specific expressed miR-375 and human heart tissue specific expressed miR-1.

8. Infectious cDNA construct according to any of the previous claims, characterized in that
the at least one or more miR-TS are present as twofold, threefold, fourfold, fivefold or more multi-fold repetitions or repetition cassettes, preferably at least twofold up to threefold repetitions or repetition cassettes.

9. Infectious cDNA construct according to any of the previous claims, characterized in that
the cDNA construct further comprises at least one or more sequence elements selected from the group consisting of: Multiple cloning site, origin of replication, selection gene, short haipin RNAs (shRNAs) and transgenes (e.g., immune system stimulating transgenes as interleukine 2 (IL-2), IL-6, IL-12 or granulocyte colony-stimulating factor (G-CSF)) or tumor toxic genes, wherein these further sequences are integrated into the backbone of the cDNA construct.

10. Infectious cDNA construct according to any of the previous claims, characterized in that
the genomic sequence of the CVB3 group virus encodes a replication competent virus, vector virus and/or viral particle.

11. Infectious cDNA construct according to any of the previous claims, characterized in that
the genomic sequence of CVB3 is selected from attenuated or aggressive CVB3 group virus strains, preferably selected from the group consisting of the strains, e.g., PD, rPD, Nancy, H3, 31-1-93, RD, P2035A, 28, HA and GA and wherein the genomic sequence of the CVB3 is defined by a nucleotide sequence of one of those strains.

12. A viral particle or vector virus comprising the cDNA construct according to any of the claims 1 to 11.

13. A pharmaceutical composition comprising the infectious cDNA according to any of the claims 1 to 11 and/or the vector virus or viral particle of claim 12 and a pharmaceutical acceptable carrier or diluent.

14. Infectious cDNA construct according to any of the claims 1 to 11, infectious viral particle or vector virus according to claim 12 or pharmaceutical composition according to claim 13 for use in the treatment of cancer and/or metastasizing cancer,
wherein the miR sequence with tissue-specific expression complementary to the at least one or more miR-TS is each highly expressed in said tissue or tissues as compared to the respective expression status in the cancer and/or metastasizing cancer, where the expression status is low or absent.

15. Infectious cDNA construct, viral particle or pharmaceutical composition for use in the treatment of cancer and/or metastasizing cancer according to claim 14,
wherein the cancer is selected from the group consisting of colorectal cancer (colon cancer), breast cancer, lung cancer, liver cancer and/or the corresponding metastases of the aforementioned cancers.


SEQUENCE LISTING (32 sequences)

SEQ ID NO: 1 - DNA, 22 bp, Artificial Sequence
  miR-375TS
tcacgcgagccgaacgaacaaa

SEQ ID NO: 2 - DNA, 22 bp, Artificial Sequence
  miR-1TS
atacatacttctttacattcca

SEQ ID NO: 3 - RNA, 74 nt, Artificial Sequence
  miR-375TS construct
ucacgcgagccgaacgaacaaauauaucacgcgagccgaacgaacaaagcguucacgcgagccgaacgaacaaa

SEQ ID NO: 4 - RNA, 102 nt, Artificial Sequence
  miR-375TS/miR-1TS-construct
ucacgcgagccgaacgaacaaauauaucacgcgagccgaacgaacaaagcgcauacauacuucuuuacauuccaaggcauauacauacuucuuuacauucca

SEQ ID NO: 5 - RNA, 72 nt, Artificial Sequence
  miR-39TS
caagcugauuuacacccgguguauacaagcugauuuacacccggugagcgucaagcugauuuacacccggug

SEQ ID NO: 6 - DNA, 116 bp, Artificial Sequence
  miR-375TS construct
aagcgatcgctcgaggataggcacctcacgcgagccgaacgaacaaatatatcacgcgagccgaacgaacaaagcgctcacgcgagccgaacgaacaaaaatgaccgtggtttaaa

SEQ ID NO: 7 - DNA, 74 bp, Artificial Sequence
  miR-375TS construct without stuffer sequence
tcacgcgagccgaacgaacaaatatatcacgcgagccgaacgaacaaagcgctcacgcgagccgaacgaacaaa

SEQ ID NO: 8 - DNA, 128 bp, Artificial Sequence
  miR-375TS/miR-1TS construct
agcgatcgctcgaggataggcacctcacgcgagccgaacgaacaaatatatcacgcgagccgaacgaacaaagcgcatacatacttctttacattccaaggcctatatacatacttctttacattcca

SEQ ID NO: 9 - DNA, 104 bp, Artificial Sequence
  miR-375TS/miR-1TS construct without stuffer sequence
tcacgcgagccgaacgaacaaatatatcacgcgagccgaacgaacaaagcgcatacatacttctttacattccaaggcctatatacatacttctttacattcca

SEQ ID NO: 10 - DNA, 7417 bp, Coxsackievirus
  CVB3 H3
ttaaaacagcctgtgggttgatcccacccacagggcctattgggcgctagcactctggtatcacggtacctttgtgcgcctgttttatatcccctcccccaactgtaacttagaagtaacacactccgatcaacagtcagcgtggcacaccagccatgttttgatcaagcacttctgttaccccggactgagtatcaatagactgctcacgcggttgaaggagaaagcgttcgttatccggccaactacttcgaaaaacccagtaacaccatagaggttgcagagtgtttcgctcagcactaccccagtgtagaccaggccgatgagtcaccgcattccccacgggcgaccgtggcggtggctgcgttggcggcctgcctatggggaaacccataggacgctctaatacagacatggtgcgaagagtctattgagctagttggtaatcctccggcccctgaatgcggctaatcctaactgcggagcacacaccctcaaaccagagggcagtgtgtcgtaacgggcaactctgcagcggaaccgactactttgggtgtccgtgtttcattttattcctatactggctgcttatggtgacaattgagagattgttaccatatagctattggattggccatccggtgtctaatagagctattatatatctctttgttggatttataccacttagcttgagagaggttaaaacattacaattcattgttaaattgaatacaacaaaatgggagctcaagtatcaacgcaaaagactggggcacatgagaccgggctgaatgctagcggcaattccattattcactacacgaatattaattattacaaagacgccgcatccaactcagccaatcggcaggatttcactcaagacccgggcaagttcacagaaccagtgaaagatatcatgattaaatcactaccagctctcaactcccccacagtagaggagtgcggatacagtgacagggtgagatcaatcacactaggtaactccaccataacgactcaggaatgcgctaacgtggtggtaggctatggagtgtggccagattatctgaaggatagcgaggctacagcagaggaccaaccgacccaaccagacgttgccacatgtaggttctatacccttgactctgtacaatggcagaaaacctcaccaggatggtggtggaagctgcctgatgctttgtcgaacttaggactgtttgggcagaacatgcagtaccactacttgggccgaactgggtataccatacatgtgcagtgcaatgcatccaagttccaccaaggatgcttgctagtagtgtgtgtaccggaagctgagatgggttgcgcaacgctaaacaacaccccatccagtgcagaattgctggggggcgatagcgccaaagagtttgcggacaaaccggttgcatccgggtccaacaagttggtacagagggtggtgtataatgcaggcatgggggtgggtgttggaaaccttaccattttccctcaccagtggatcaatctacgcaccaacaatagtgctacaattgtgatgccatacaccaacagcgtacctatggataacatgtttaggcataacaacgtcaccctaatggttatcccatttgtaccgctagattactgccctgggtctaccacgtacgtcccaatcacgatcacgatagccccaatgtgtgccgagtacaatggactacgtttggccgggcaccagggcttaccaaccatgaacactccggggagctgtcaatttctgacatcagacgacttccaatcaccatctgccatgccgcaatacgacgtcacgccagagatgaggatacctggtgaggtgaagaacttgatggaaatagctgaggttgactcagttgtcccggtccaaaatgttggagagaaggtcaactccatggaagcgtaccagatacctgtgagatccaatgaaggatctggaacgcaagtattcggcttcccactgcaaccagggtattcgagtgttttcagtcggacgctcctaggagagatcttgaactattacacccattggtcaggcagcataaagcttacgtttatgttctgtggttcggccatggccactggaaaattccttttggcatactcaccaccaggcgctggggctcccacaaaaagggttgatgctatgcttggcactcatgtagtttgggatgtggggctacaatcaagttgcgtgctgtgcataccctggataagccaaacacactaccggtatgttgcttcagatgagtataccgcagggggttttattacgtgctggtatcaaacaaacatagtcgtcccagcagatgcccaaagctcctgttacatcatgtgtttcgtatcagcatgcaatgatttctctgtcaggctattgaaggacactccttttatttcgcagcaaaactttttccagggccccgtggaagacgcgataacagccgccatagggagagttgcggacaccgtgggtacagggccaaccaactcagaggctataccagcactcactgctgctgagacaggtcacacgtcgcaagtagtgccgagtgacaccatgcagacacgccacgttaagaactaccattcaaggtctgagtcgaccatagagaacttcctatgtaggtcagcatgcgtgtactttacagagtatgaaaactcaggcgccaagcggtatgctgaatgggtaataacaccacgacaagcggcacaacttaggagaaagctagaattctttacctacgtccggtttgacctggagctgacgtttgtcataacaagtactcaacagccctcaaccacacagaaccaggatgcacagatcctaacacaccaaattatgtatgtaccaccaggtgggcctgtgccagacaaagtcgattcttacgtgtggcaaacatctacgaatcccagtgtgttttggaccgagggaaacgccccgccgcgtatgtccgtaccgtttttgagcattggcaacgcttattcaaatttctatgatgggtggtctgaattttccaggaacggggtttacggtatcaacacactaaacaacatgggcacgctatatgcaagacatgtcaatgctggaagcacgggaccaataaaaagcaccattagaatctacttcaaacctaagcatgtcaaagcgtggatacctagaccacctagactctgccaatacgagaaggcaaagaacgtgaacttccaacccagcggagttaccactactaggcaaagcatcactacaatgacaaatacgggcgcatttggacaacaatcaggggcagtatacgtagggaactacagggtagtaaatagacatctagctaccagtgctgactggcaaaactgtgtgtgggaaaattacaacagagacctcttagtgagcacgaccacagcacatggatgtgatattatagccagatgtcggtgtacaacgggagtgtacttttgtgcgtccaaaaacaaacactacccaatttcatttgaaggaccaggtatagtagaggtccaagagagtgagtactaccctaggagataccaatcccatgtgcttttagcagctgggttttccgaaccaggtgactgtggcggtatcctaaggtgtgagcatggtgtcattggcattgtgaccatggggggtgaaggcgtggtcggctttgcagacatccgtgatctcctgtggctggaagatgatgcaatggaacagggagtgaaggactatgtggaacagcttggaaatgcattcggctctggcttcactaaccaaatatgtgagcaagtcaacctcctgaaagaatcactagtgggtcaagactccatcttagagaagtctctaaaagccttagttaagataatatcagccttagtaattgtggtgaggaaccacgatgacctaatcacggtgactgccacactagccctcattggttgtacctcgtccccatggcggtggcttaagcagaaagtgtcccaatattacggaatacccatggctgaacgccaaaacaacggatggctaaaaaagttcactgagatgacaaacgcctgcaagggcatggaatggatagccattaagattcagaaattcattgagtggctcaaagttaaaattttacctgaggtcagggaaaaacacgaattcctgaacagactcaaacagctccccctgttagagagtcaaattgccacaatcgagcaaagtgcaccgtcacagagtgaccaggagcaattgttttccaatgtccaatactttgctcactattgcagaaagtatgctcccctttatgcatcagaggcaaagagagtgttctcccttgagaagaagatgagtaattacatacagttcaagtccaaatgccgtattgagcctgtatgtctgctcctgcatgggagtcccggtgcaggtaagtcagttgcaacaaatctgatcggaagatcactcgcggaaaagttaaacagctcagtgtattcactacccccagacccagatcacttcgatggctataaacagcaggctgtagtgatcatggacgatctatgtcagaaccccgatgggaaagatgtctccttgttctgtcagatggtttccagtgtggattttgtaccacccatggccgccctggaagagaaaggcatcttgttcacctccccgttcgttttggcatcaaccaatgcgggatctattaacgctccaactgtgtcagacagcagggccttagcaaggagattccactttgacatgaatattgaagttatttctatgtacagccaaaatggcaagataaacatgccaatgtcagtgaagacgtgtgatgaagagtgttgcccagtcaactttaagaaatgttgcccgttagtctgtggaaaggccatccaattcatagacagaagaactcaagtcagatactccctcgatatgctggtaactgagatgtttagggaatacaaccacaggcacagtgtcggggctacccttgaggcactgttccagggtccaccagtatacagagagattaagattagcgtggcaccagaaacaccaccaccaccagctatcgcggacttgcttaaatcagtggatagcgaagccgtgagagagtattgcaaagaaaagggatggttggttcctgaggtcaactccaccctccaaattgaaaaacatgtcagtcgggctttcatctgcttgcaggcaataactacgtttgtgtcagtagctggaatcatctatataatatacaagctctttgcaggctttcaaggtgcatatacaggaatacccaaccagaagcccaaggtacctaccctaaggcaagcaaaagtgcagggtcctgcatttgaatttgctgttgcaatgatgaagaggaactcaagcacagtgaagacagagtatggtgagttcaccatgttgggcatttatgataggtgggccgttttgccacgtcatgccaaacccggaccaaccatcctgatgaatgaccaggaggtaggcgtgctggacgctaaagagttagtggataaggatggtacaaacctagaactgacactgctcaagttgaacaggaacgagaagttcagagacatcagaggcttcttagcaaaggaggaggtggaagtcaacgaggccgtgctagcaattaataccagtaaatttcccaacatgtacattccggtgggacaagtcacggattacggtttcctaaacctgggtggtacgcccactaaaagaatgcttatgtacaacttccccacgagagcaggtcaatgtggcggagtactcatgtccaccggcaaagtcctggggatccatgttggtggaaatggtcatcaaggtttctcagcggcacttctcaagcactatttcaatgatgaacaaggagagatcgagtttattgagagctcaaaggaagcagggttccctattatcaacacacctagtaagactaagctggagccgagtgtcttccaccaggtttttgaaggtgacaaagagccagcggtcctcaggaatggtgatccacgcctcaaagtcaactttgaggaggccatattttccaagtacatcgggaatgttaacacacacgtggatgaatacatgatggaggctgttgaccattatgccggacaattggccaccctagacattagcactgaaccaatgaagttggaggatgctgtatacggtactgaaggccttgaggctcttgatctaacaacgagtgcaggttacccttatgtcgccctgggcatcaagaagagagacatcctctcaaagaagaccagggaccttactaagctgaaagagtgcatggataagtacggtctaaacctaccaatggtaacctatgtgaaagacgaactcagatctgcagagaaggtggcaaagggaaagtccaggctcattgaggcgtccagtttgaatgactctgtggcaatgagacagacattcggcaacttgtacaaaacttttcacctaaacccagggattgtgactggcagtgctgtcgggtgtgacccggacctcttttggagtaaaataccagtgatgttggacggtcatctcatagcttttgattattctggatatgatgctagcttgagtcccgtatggtttgcttgtttaaaactactacttgaaaaacttggttactcgcacaaggagaccaattacattgattacctgtgcaactcccatcacctgtacagggacaaacattattttgtgcggggtggcatgccatctggatgttctggcacaagcatctttaactcaatgataaataacatcataatcaggacactcatgctgaaggtgtacaaagggatcgacttggatcaattcaggatgattgcttatggtgacgatgtgattgcatcatacccgtggcccatagatgcgtctttgcttgctgaagctggcaaggactatggattaatcatgacaccagcagacaaaggggagtgcttcaatgaagttacttggactaacgtcacattcctaaagaggtattttagagcagatgaacaatacccctttttagtgcaccccgttatgcccatgaaagacatacacgaatcaatcagatggaccaaggatccaaagaatacccaagaccatgtgcgctcattgtgcttattggcctggcacaacggggagcacgaatatgaggagtttatccgcaaaatcaggagcgtcccagttggacgttgtttgactctacctgcgttctcaaccttacgtaggaagtggttggactctttctaaattagagacaatttgatctgatttgaattggcttaaccctactgtactaaccgaactagacaacggtgcagtaggggtaaattctccgcattcggtgcggaaaaaaaaaaaaaaaaa

SEQ ID NO: 11 - DNA, 7415 bp, Coxsackievirus
  CVB3 rPD
ttaaaacagcctgtgggttgatcccacccacagggcccattgggcgctagcactctggtatcacggtacctttgtgcgcctgttttatacccccccccccaactgtaacttagaagcaacacacaccgatcaacagtcagcgtggcacaccagccacgttttgatcaagcacttctgttaccccggactgagtatcaatagactgctcacgcggttgaaggagaaagcgttcgttatccggccaactatttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagattgttaccatatagctattggattggccatccggtgaccaatagagctattatatatctctttgttgggtttataccacttagcttgaaagaggttaaaacattacaattcattgttaagttgaatacagcaaaatgggagctcaagtatcaacgcaaaagactggggcacatgagaccgggctgaatgctagcggcaattccatcattcactacacaaatattaattattacaaggatgccgcatccaactcagccaatcggcaggatttcgctcaagacccgggcaagttcacagaaccagtaaaagatatcatgattaaatcactaccagctctcaactcccccacagtagaggagtgcggatacagtgacagggtgagatcaatcacattaggtaactccaccataacgactcaggaatgcgccaacgtggtggtgggctatggagtatggccagattatctaaaggatagtgaggcaacagcagaggaccaaccgacccaaccagacgttgccacatgtaggttctatacccttgactctgtgcaatggcagaaaacctcaccaggatggtggtggaagctgcccgatgctttgtcgaacttaggactgtttgggcagaacatgcagtaccactacttaggccgaactgggtataccgtacatgtgcagtgcaatgcatctaagttccaccaaggatgcttgctagtagtgtgtgtaccggaagctgagatgggttgcgcaacgctagacaacaccccatccagtgcagaattgctggggggcgatagcgcaaaagagtttgcggacaaaccggtcgcatccgggtccaacaagttggtacagagggtggtgtataatgcaggcatgggggtgggtgttggaaacctcaccattttcccccaccaatggatcaacctacgcaccaataatagtgctacaattgtgatgccatacaccaacagtgtacctatggataacatgtttaggcataacaacgtcaccctaatggttatcccatttgtaccgctagattactgccctgggtccaccacgtacgtcccaattacggtcacgatagccccaatgtgtgccgagtacaatgggttacgtttagcagggcaccagggcttaccaaccatgaatactccggggagctgtcaatttctgacatcagacgacttccaatcgccatccgccatgccgcaatatgacgtcacaccagagatgaggatacctggtgaggtgaaaaacttgatggaaatagctgaggttgactcagttgtcccagtccaaaatgttggagagaaggtcaactctatggaagcataccagatacctgtgagatccaatgaaggatctggaacgcaagtattcggctttccactgcaaccagggtactcgagtgtttttagtcggacgctcctaggagagatcttgaactattatacacattggtcaggcagcataaagcttacgtttatgttctgtggttcggccatggctactggaaaattccttttggcatactcaccaccaggtgctggagctcctacaaaaagggttgatgccatgcttggtactcatgtagtttgggacgtggggctacaatcaagttgcgtgctgtgtataccctggataagccaaacacactaccggtatgttgcttcagatgagtataccgcagggggttttattacgtgctggtatcaaacaaacatagtggtcccagcggatgcccaaagctcctgttacatcatgtgttttgtgtcagcatgcaatgacttctctgtcaggctattgaaggatactcctttcatttcgcagcaaaacttttaccagggcccagtggaagacgcgataacagccgctatagggagagttgcggataccgtgggtacagggccaaccaactcagaagctataccagcactcactgctgctgagacaggtcacacgtcacaagtagtgccgggtgacaccatgcagacacgccacgttaagaactaccattcaaggtccgagtcaaccatagagaacttcctatgtaggtcagcatgcgtgtactttacgaagtatgcaaactcaggtgccaagcggtatgctgaatgggcaataacaccacgacaagcagcacaacttaggagaaagctagaattctttacctacgtccggttcgacctggagctgacgtttgtcataacaagtactcaacagccctcaaccacacagaaccaagacgcacagatcctaacacaccaaattatgtatgtaccaccaggtggacctgtaccagagaaagttgattcatacgtgtggcaaacatctacgaatcccagtgtgttttggaccgagggaaacgccccgccgcgcatgtccataccgtttttgagcattggcaacgcctattcaaatttctatgacggatggtctgaattttccaggaacggagtttacggcatcaacacgctaaacaacatgggcacgctatatgcaagacatgtcaactctggaagcacgggtccaataaaaagcaccattagaatctacttcaaaccgaagcatgtcaaagcgtggatacctagaccacctagactctgccaatacgagaaggcaaagaacgtgaacttccaacccagcggagttaccactactaggcaaagcatcactacaatgacaaatacgggcgcatttggacaacaatcaggggcagtgtatgtggggaactacagggtagtaaatagacatctagctaccagtgctgactggcaaaactgtgtgtgggaaagttacaacagagacctcttagtgagcacgaccacagcacatggatgtgatattatagccagatgtcagtgcacaacgggagtgtacttttgtgcgtccaaaaacaagcactacccaatttcgtttgaaggaccaggtctagtagaggtccaagagagtgaatactaccccaggagataccaatcccatgtgcttttagcagctggattttccgaaccaggtgactgtggcggtatcctaaggtgtgagcatggtgtcattggcattgtgaccatggggggtgaaggcgtggtcggctttgcagacatccgtgatctcctgtggctggaagatgatgcaatggaacagggagtgaaggactatgtggaacagcttggaaatgcattcggctccggctttactaaccaaatatgtgagcaagtcaacctcctgaaagaatcactagtgggtcaagactccatcttagagaaatctctaaaagccttagttaagataatatcagccttagtaattgtggtgaggaaccacgatgacctgatcactgtgactgccacactagcccttatcggttgtacctcgtccccgtggcggtggctcaaacagaaggtgtcacaatattacggaatccctatggctgaacgccaaaacaatagctggcttaagaaatttactgaaatgacgaatgcttgcaagggtatggaatggatagctgtcaaaattcagaaattcattgaatggctcaaagtaaaaattttgccagaggtcagggaaaaacacgaattcctgaacagacttaaacaactccccttattagaaagtcagatcgccacaatcgagcagagcgcgccatcccaaagtgaccaggaacaattattttccaatgtccaatactttgcccactattgcagaaagtacgctcccctctatgcagctgaagcaaagagggtgttctcccttgagaagaagatgagcaattacatacagttcaagtccaaatgccgtattgaacctgtatgtttgctcctgcacgggagccctggtgccggcaagtcggtggcaacaaacttaattggaaggtcgcttgctgagaaactcaacagctcagtgtactcactaccgccagacccagatcacttcgacggatacaaacagcaggccgtggtgattatggacgatctatgccagaatcctgatgggaaagacgtctccttgttctgccaaatggtttccagtgtagattttgtaccacccatggctgccctagaagagaaaggcattctgttcacctcaccgtttgtcttggcatcgaccaatgcaggatctattaatgctccaaccgtgtcagatagcagagccttggcaaggagatttcactttgacatgaacatcgaggttatttccatgtacagtcagaatggcaagataaacatgcccatgtcagtcaagacttgtgacgatgagtgttgcccggtcaattttagaaagtgctgccctcttgtgtgtgggaaggctatacaattcattgatagaagaacacaggtcagatactctctagacatgctagtcaccgagatgtttagggagtacaatcatagacatagcgtggggaccacgcttgaggcactgttccagggaccaccagtatacagagagatcaaaattagcgttgcaccagagacaccaccaccgcccgccattgcggacctgctcaaatcggtagacagtgaggctgtgagggagtactgcaaagaaaaaggatggttggttcctgagatcaactccaccctccaaattgagaaacatgtcagtcgggctttcatttgcttacaggcattgaccacatttgtgtcagtggctggaatcatatatataatatataagctctttgcgggttttcaaggtgcttatacaggagtgcccaaccagaagcccagagtgcctaccctgaggcaagcaaaagtgcaaggccctgcctttgagttcgccgtcgcaatgatgaaaaggaactcaagcacggtgaaaactgaatatggcgagtttaccatgctgggcatctatgacaggtgggccgttttgccacgccacgccaaacctgggccaaccatcttgatgaatgatcaagaggttggtgtgctagatgccaaggagctagtagacaaggacggcaccaacttagaactgacactactcaaattgaaccggaatgagaagttcagagacatcagaggcttcctagccaaggaggaagtggaggttaatgaggcagtgctagcaattaacaccagcaagtttcccaacatgtacattccagtaggacaggtcacagaatacggcttcctaaacctaggtggcacacccaccaagagaatgcttatgtacaacttccccacaagagcaggccagtgtggtggagtgctcatgtccaccggcaaggtactgggtatccatgttggtggaaatggccatcagggcttctcagcagcactcctcaaacactacttcaatgatgagcaaggtgaaatagaatttattgagagctcaaaggacgccgggtttccagtcatcaacacaccaagtaaaacaaagttggagcctagtgttttccaccaggtctttgaggggaacaaagaaccagcagtactcaggagtggggatccacgtctcaaggccaattttgaagaggctatattttccaagtatataggaaatgtcaacacacacgtggatgagtacatgctggaagcagtggaccactacgcaggccaactagccaccctagatatcagcactgaaccaatgaaactggaggacgcagtgtacggtaccgagggtcttgaggcgcttgatctaacaacgagtgctggttacccatatgttgcactgggtatcaagaagagggacatcctctctaagaagactaaggacctaacaaagttaaaggaatgtatggacaagtacggcctgaacctaccaatggtgacttatgtaaaagatgagctcaggtccatagagaaggtagcgaaaggaaagtctaggctgattgaggcgtccagtttgaatgattcagtggcgatgagacagacatttggtaatctgtacaaaactttccacctaaacccaggggttgtgactggtagtgctgttgggtgtgacccagacctcttttggagcaagataccagtgatgttaaatggacatctcatagcatttgattactctgggtacgatgctagcttaagccctgtctggtttgcttgcctaaaaatgttacttgagaagcttggatacacgcacaaagagacaaactacattgactacttgtgtaactcccatcacctgtacagggataaacattactttgtgaggggtggcatgccctcgggatgttctggtaccagtattttcaactcaatgattaacaacatcataattaggacactaatgctaaaagtgtacaaagggattgacttggaccaattcaggatgatcgcatatggtgatgatgtgatcgcatcgtacccatggcctatagatgcatctttactcgctgaagctggtaagggttacgggctgatcatgacaccagcagataagggagagtgctttaacgaagttacctggaccaacgtcactttcctaaagaggtattttagagcagatgaacagtaccccttcctggtgcatcctgttatgcccatgaaagacatacacgaatcaattagatggaccaaggatccaaagaacacccaagatcacgtgcgctcactgtgtttattggcttggcataacggggagcacgaatatgaggagttcatccgtaaaattagaagcgtcccagtcggacgttgtttgaccctccccgcgttttcaactctacgcaggaagtggttggactccttttagattagagacaatttgaaataatttagattggcttaaccctactgtgctaaccgaaccagataacggtacagtaggggtaaattctccgcattcggtgcggaaaaaaaaaaaaaaa

SEQ ID NO: 12 - RNA, 22 nt, Artificial Sequence
  mature hsa-miRNA-375
uuuguucguucggcucgcguga

SEQ ID NO: 13 - RNA, 22 nt, Artificial Sequence
  miR-375TS
aaacaagcaagccgagcgcacu

SEQ ID NO: 14 - RNA, 22 nt, Artificial Sequence
  mature hsa-miR-1
uggaauguaaagaaguauguau

SEQ ID NO: 15 - RNA, 22 nt, Artificial Sequence
  miR-1TS
accuuacauuucuucauacaua

SEQ ID NO: 16 - RNA, 101 nt, Artificial Sequence
  Consensus miR-375TS construct
uaaagcgaucgcucgaggauaggcaccucacgcgagccgaacgaacaaauauaucacgcgagccgaacgaacaaagcgcucacgcgagccgaacgaacaaa

SEQ ID NO: 17 - RNA, 101 nt, Artificial Sequence; identical to SEQ ID NO: 16
  H3N-375TS, 32 days pi (post implantation of) tumor 1

SEQ ID NO: 18 - RNA, 101 nt, Artificial Sequence
  H3N-375TS, 32 days pi (post implantation of) tumor 2
uaaagcgaucgcucgaggauaggcaccucacccgagccagucgaacaaauauaucacgcgagccgaacgaacaaagcgcucacgcgagccgaacgaacaaa

SEQ ID NO: 19 - RNA, 101 nt, Artificial Sequence
  H3N-375TS, 32 days pi (post implantation of) tumor 3
uaaagcgaucgcucgaggauaggcaccucacguuagccgaacgaacaaauauaucacgcgagccgaacgaacaaagcgcucacgcgagccgaacgaacaaa

SEQ ID NO: 20 - RNA, 127 nt, Artificial Sequence
  Consensus miR-375TS/miR-1TS
uaaagcgaucgcucgaguaggcaccucacgcgagccgaacgaacaaauauaucacgcgagccgaacgaacaaagcgcauacauacuucuuuacauuccaaggcauauacauacuucuuuacauucca

SEQ ID NO: 21 - RNA, 127 nt, Artificial Sequence; identical to SEQ ID NO: 20
  H3N- miR-375TS/miR-1TS, 32 days pi (post implantation of) tumor 1

SEQ ID NO: 22 - RNA, 127 nt, Artificial Sequence; identical to SEQ ID NO: 20
  H3N- miR-375TS/miR-1TS, 32 days pi (post implantation of) tumor 2

SEQ ID NO: 23 - RNA, 127 nt, Artificial Sequence
  H3N- miR-375TS/miR-1TS, 32 days pi (post implantation of) tumor 3
uaaagcgaucgcucgaguaggcaccucacgcgagccgaacgaacaaauauaucacgcgagccgaacgaacaaagcgcauacauacuuccuuacauuccaaggcauauacauacuucuuuacauucca

SEQ ID NO: 24 - DNA, 59 bp, Artificial Sequence
  miR-1TS sense primer
tccaaggcctatatacatacttctttacattccattagagacaatttgatctgatttga

SEQ ID NO: 25 - DNA, 52 bp, Artificial Sequence
  miR-1TS antisense primer
tatataggccttggaatgtaaagaagtatgtatgcgctttgttcgttcggct

SEQ ID NO: 26 - DNA, 19 bp, Artificial Sequence
  RT antisense primer
ctactgcaccgttgtctag

SEQ ID NO: 27 - DNA, 19 bp, Artificial Sequence
  sense primer
ccatagatgcgtctttgct

SEQ ID NO: 28 - DNA, 18 bp, Artificial Sequence
  antisense primer
ccgttgtctagttcggtt

SEQ ID NO: 29 - DNA, 18 bp, Artificial Sequence
  Sequencing primer
caggagcgtcccagttgg

SEQ ID NO: 30 - DNA, 25 bp, Artificial Sequence
  Stuffer 5- (part of SEQ ID NO6)
aagcgatcgctcgaggataggcacc

SEQ ID NO: 31 - DNA, 17 bp, Artificial Sequence
  Stuffer 3- (part of SEQ ID NO6)
aatgaccgtggtttaaa

SEQ ID NO: 32 - DNA, 24 bp, Artificial Sequence
  Stuffer 5- (part of SEQ ID NO8)
agcgatcgctcgaggataggcacc